(19) United States
(12) Patent Application Publication
(10) Pub. No.: US 2006/0057561 A1
(43) Pub. Date: Mar. 16, 2006

(54) Virus detection method, primers therefor and screening kit

(76) Inventor: Keith W Hart, Brecon (GB)

Correspondence Address: KING & SCHICKLI, PLLC, 247 NORTH BROADWAY, LEXINGTON, KY, 40507, US

(21) Appl. No.: 10/480,852

(22) PCT Filed: Jun. 13, 2002
(86) PCT No.: PCT/GB02/02847

§ 371 Date: Jul. 26, 2004

(30) Foreign Application Priority Data
Jun. 14, 2001 (GB) 0114430.2
Mar. 28, 2002 (GB) 0207276.7

(51) Int. Cl. C12Q 1/70; C12P 19/34

(52) U.S. Cl. 435/5; 435/91.2

(57) ABSTRACT

Discloses a novel detection and typing method for viruses, such as human papillomaviruses, based on real-time PCR using self-probing amplicon fluorescent primers. The method comprises: (IA) contacting the sample with a self-probing amplicon (‘virus self-probing amplicon’) comprising (i) a virus primer capable of hybridising to at least one target viral nucleic acid sequence and undergoing amplification thereof under primer amplification conditions to form a virus primer extension product; (ii) a virus probe comprising a nucleic acid sequence complementary to a target sequence of the virus primer extension product and capable of hybridisation thereto, provided that the self-probing amplicon is adapted to ensure that the virus probe is unresponsive to amplification under the primer amplification conditions; and (iii) a member of a virus signalling system, which system is capable of causing a detectable signal to be effected on hybridisation of the virus probe sequence to the virus primer extension product, whereby presence or absence of the target viral nucleic acid sequence in the sample is indicated by the detectable signal; (IB) amplifying the product of step (IA) under the primer amplification conditions to an extent enabling the detectable signal to be effected after step(II); and (II) separating the virus primer extension product from the target viral nucleic acid sequence; allowing the virus probe to hybridise to the target sequence of the virus primer extension product; and monitoring the signalling system. This method is quick, simple, specific, sensitive, and capable of estimating viral load per cell. The results of over 100 HPV typing reactions performed on cell lines, biopsies and cervical cytobrush samples are given which, when compared to the current reference HPV detection and typing technique, present a kappa value of 0.89. The method is also applicable to other viruses, such as SV40.

The present invention relates to an improved method for detecting, typing and characterising the presence of viruses, particularly those that have been associated with carcinogenic activity in mammals, such as human papillomavirus and SV40, and to primers and probes for use in the method. The invention further relates to a diagnostic kit and screening method, which uses the kit.

As well as there being various viruses that have been associated with carcinogenic effects and therefore there being a need to be able to identify the presence of such viruses in clinical samples, it is also important to be able accurately to determine the viral load per cell and integration status of the virus in the affected organism (most importantly, in humans). Accordingly, it is desired to provide means for establishing the infecting virus type, the viral load (per cell ie the number of viral copies per cell or ratio of viral genome:human genome) and the integration state (ie whether the viral DNA is ‘free’ in the cytosol of the cell or integrated into the host genome) of the virus, since these factors are known to have profound implications for patient prognosis.

These issues will first be illustrated with reference to two particular virus categories: human papillomaviruses (HPV) and SV40.

Cervical cancer is the second most frequent cause of death from cancer in women, worldwide. Cervical screening programmes reduce the incidence of cervical cancer; however, 50% of invasive cervical cancers arise in women screened using existing cytological methodologies. In recent years, it has been established that a subset of human papillomaviruses (HPV) are associated with cervical cancer, and it is estimated that HPV DNA is present in over 99% of these cancers. There are currently 84 types of HPV, around 30 of which infect the genital tract. Therefore, HPV detection and typing techniques have been proposed as an adjunct to, or replacement for, the current cytological screening regime. Clearly, the success of such strategies will depend on the development of rapid, reliable, sensitive and specific HPV-detection methods applicable in the clinical setting.

Currently, there are eight main approaches to the detection and typing of HPV, all of which display advantages and disadvantages, depending on application. These are summarised in Table 1, from which it can be seen that no single technique performs optimally in both clinical and research settings. By way of background, the PCR method has been introduced as the most sensitive method for the detection of HPV DNA in clinical specimens. However, a significant heterogeneity at the nucleotide level is found between the different HPV genotypes. This has hampered the development of a simple, universal PCR test for the detection of all HPV genotypes. Despite this, HPV PCR methods have been developed, allowing the detection of a broad spectrum of mainly mucosotropic HPV genotypes.

TABLE 1
Current HPV Detection and Typing Techniques.

| | Analysis | Comments | | |
|---|---|---|---|---|
| Technique | Methodology | Advantages | Disadvantages | Reference |
| PCR-based | | | | |
| Consensus | | | | |
| Primers | | | | |
| | Enzyme-Linked | Accurate, | Laborious, | Jacobs et al J Clin |
| | Immunosorbent Assay | sensitive | expensive. | Microbiol. 35 791-5 |
| | | | | (1997) |
| | Restriction Fragment | Quick, cheap | Non-quantitative, | Rodu et al in |
| | Length Polymorphism | | low sensitivity. | Biotechniques 10 632-7 |
| | | | | (1991) |
| | Dot blot | Quick, accurate | Limited resolution, | Gravitt et al J Clin |
| | | | non-quantitative. | Microbiol 36 3020-7 |
| | | | | (1998) |
| | Sequencing | Accurate, | Laborius, expensive. | Vernon et al J Clin |
| | | sensitive | Multiple types | Microbiol 38 651-5 |
| | | | problematic | (2000) |
| | Taqman ™ | Accurate, | Complex, expensive. | Swan et al J Clin |
| | probes | sensitive, | | Microbiol 35 886-91 |
| | | quantitative | | (1997) |
| Type- | Agarose gel | Quick, cheap | Non-quantitative, | Evander et al in Arch |
| specific | electrophoresis | | low sensitivity. | Virol 116 221-33 (1991) |
| primers | | | | |
| Non-PCR | | | | |
| based | | | | |
| Hybrid | Enzyme Linked | Semi-quantitative, | Low sensitivity, | Vernon et al J Clin |
| Capture ™ | Immunosorbent | commercially | specificity and | Microbial 38 651-5 |
| | Assay | available | resolution. | (2000) |

HPV is genetically composed of early (E) and late (L) genes, which are functionally divided into several open reading frames (ORFs): viral replication (E1), regulation of transcription (E2), coding for cytoplasmic proteins (E4) and malignant transformation (E5, E6, E7) as early genes, and coding for capsid proteins (L1, L2) as late genes.

A combination of the general primers GP5 and GP6, originally selected from the HPV L1 region on the basis of sequence information from HPV-6, -11, -16, -18, -31 and -33, was found to amplify target DNA of at least 27 mucosotropic HPV genotypes under conditions that allow mismatch acceptance. The general primers GP5 (5′-TTT GTT ACT GTG GTA GAT AC-3′) and GP6 (3′-ACT AAA TGT CAA ATA AAA AG-5′) (Snijders etal, J. Gen. Virol. 71 173-181 (1990)), which span a region of 140-150 bp from the L1 open reading frame of a broad spectrum of HPV genotypes, were used in general primer-mediated PCR. The strength of this (GP-PCR) method has been further substantiated by the detection of HPV DNA in 100% of cervical scrapes cytomorphologically classified as Pap IV (carcinoma in situ) and Pap V (carcinoma) in the Netherlands. This suggests that, in the Dutch population, all genital high risk HPV's can be detected by this assay.

But, using GP-PCR in routine diagnostic practice, it has been found that a small number of clinical samples give rise to ambiguous results, reflected by weak GP-PCR signals accompanied by a relatively high background of co-amplified cellular DNA. It was then found that increased primer length contributes to more efficient amplification, and so the GP5 and GP6 primers were elongated with highly conserved sequences at the 3′ ends. When the use of these GP5+ and GP6+ primers in the PCR was compared with the original GP5 and GP6 PCR on 1 ng cloned DNA of various HPV types, it was found that the elongated general primers were significantly more sensitive.

In order to facilitate PCR-based HPV detection and typing, a colorimetric microtitre plate based hybridisation assay was developed. The method used one biotinylated primer (bio-GP6+) in the GP-PCR. Biotinylated PCR products were captured on streptavidin-coated microtitre plates, denaturated and hybridised to digoxigenin-(DIG-) labelled HPV-specific internal oligonucleotide probes. The DIG-labelled hybrids were detected using an enzyme immunoassay (EIA). In a development of this technique, two cocktails of DIG-labelled HPV type-specific oligonucleotide probes and an EIA have been used as a basis to develop a group-specific detection method for 14 high-risk (types 16, 18, 31, 35, 39, 45, 51, 52, 56, 58, 59, 66 and 68) and 6 low-risk (types 6, 11, 40, 42, 43 and 44) HPVs following a general primer GP5+/bioGP6+-mediated PCR. This technique is hereinafter referred to as PCR-EIA. The sensitivity of this high-risk/low risk (HR/LR) HPV PCR-EIA ranged from 10 to 200 HPV copies, depending on the HPV type.

Some fluorogenic probe assays for HPV DNAs are based on the PCR amplification of a portion of the L1 open reading frames of HPV-16, -18, -31, -33, and -35 DNAs by using genotype-specific probes that bind to the amplified DNA. In the TaqMan assay, the probes are blocked at their 3′ termini and hence cannot be extended by the polymerase. If, during the course of primer extension, Taq polymerase encounters a bound probe, its 5′→3′ exonuclease activity degrades the probe, releasing the 5′ fluor from the 3′ quencher. This causes an increase in the fluorescence emitted by the reporter which, in the presence of an excess amount of the probe, is directly related to the amount of HPV DNA present in the sample before amplification. ‘Molecular beacons’ comprise a probe flanked by a hairpin loop that holds a fluorophore and quencher in close proximity until specific binding of the probe to its target opens out the structure, producing a fluorescent signal.

In general, HPV DNA is present as an episomal form in cervical intra-epithelial neoplasia (CIN) lesions, and the principal form of the viral DNA in invasive cancers is integrated into the host genome. In most cervical carcinomas, HPV genomes are integrated into host cell chromosomes and transcribed into mRNAs encompassing viral and cellular sequences. In contrast, in early pre-neoplastic lesions, HPV genomes persist as episomes, and derived transcripts contain exclusively viral sequences. Thus, detection of HPV transcripts derived from integrated HPV genomes may specifically indicate both CIN lesions at high risk for progression as well as invasive cervical cancers.

When the HPV DNA becomes integrated into the cellular chromosome, generally some part of the E2 ORF is lost, releasing suppression of E6 and E7 mRNA expression. The E6 and E7 proteins can activate some oncogenes and/or inactivate host tumour suppressor gene products (p53, pRB), resulting in uncontrolled cellular growth and malignant transformation.

Integration of HPV DNA is therefore considered an important genetic change in the process of cervical carcinogenesis, and being able to determine the level of integration is believed to be important, diagnostically. Some detection techniques therefore focus on identifying and quantifying disruptions in the early genes, particularly E1/E2 and E6/E7. These have been carried out using techniques such as multiplex PCR (where primers for each sequence are included in the same reaction) and RT-PCR that allows discrimination of HPV mRNAs derived from integrated and episomal viral genomes.

Hitherto-known techniques therefore require a plurality of techniques and/or probes to detect HPV types and integration status, which is time-consuming, slow and generally unsatisfactory for clinical use. Furthermore, none of these techniques satisfactorily deal with the determination of viral load, since none result in a measure of the number of viral copies per cell. These systems have used different methods for the quantitation of virus copy number and cell copy number and thus the two are not strictly comparable. Previous methods incorporating assays for the β-globin or other housekeeping genes of the genomic DNA have, at most, given rise to a range of viral DNA copies per microgram of cellular DNA, rather than per cell. Other uses of housekeeping genes have been as PCR controls or to determine PCR limits and not to provide information about viral load.

Another disease caused by HPV is Recurrent Respiratory Papillomatosis (RRP), which is the most common benign tumour of the larynx, and is a serious condition with no satisfactory treatment and around 2-7% malignant transformation. RRP is caused by one of two HP viruses, namely, HPV-6 and HPV-11. However, these viruses can also be detected in the oropharynx of over 50% of normal school-age children and so host factors are clearly important.

It is postulated that HPV transmission is mediated during transit through the birth canal in juvenile onset RRP and during oro-genital contact in adult onset RRP. Patients with the disease have no obvious immune deficit.

Another example of a virus that has been linked to cancer, particularly mesotheliomas (cancers affecting the mesothelial cells in the lining of the chest and lung) is Simian virus 40 (SV40), named after its origin in the Simian monkey. It was discovered as a contaminant in early doses of polio vaccine and was initially thought to be harmless in humans. It was found to cause tumours in laboratory animals and since then has been linked to osteosarcomas, pituitary, thyroid, brain and neurological tumours (eg glioblastomas, astrocytomas, ependymomas and pappillomas of the choroids plexus). SV40 is now believed to be more tumorigenic thatn HPV, since one copy of SV40 per cell is believed to be capable of transforming the cell. Nevertheless, it is understood that mesotheliomas associated with SV40 can take up to 20 to 40 years to develop, but is very pernicious, causing death in about 18 months, with about 3,000 victims per annum in the USA alone.

The highly tumorigenic nature of SV40 is attributed to the fact that it produces very small amounts of ‘large T-antigen’, which can knock out both the p53 and Rbs regulatory pathways simultaneously, whereas HPV has to produce two agents (E6 and E7) to do so. SV40 also damages chromosomes by re-arrangement of DNA. Accordingly, there is a need for a method that can accurately detect, type and quantify (load per cell and integration status) of SV40, too.

The PCR technique originally used for the detection of SV40-like sequences in ependymoma and choroids plexus tumours has become the principle method used by the majority of workers in the field to study the presence of SV40 in human pleural mesothelioma. This method comprises extraction of the DNA from a sample of the tumour, and PCR amplification thereof with primers designed both to check the quality of the extracted DNA and then the presence in it of any SV40 DNA-like sequences.

The primers (SV.for 3/SV.rev) most frequently used have been designed to amplify the sequence that codes for Rb, p107 and Rb2/p130 binding domains of the large T-antigen. The incidence rates of SV40 detection using this technique in the hands of various workers range from 0% to 100% (see Jasani et al in Fron in Biosci 6 e12-22 (2001)), which may at least in part be due to the varying efficiency of the DNA extraction and PCR amplification and product detection methods.

In terms of detection of viral load, there have also been mixed results, ranging from 1 to 100 copies of SV40 genome copies per PCR reaction, and it is unclear whether the lower end of this range includes viral copies per cell rather than per PCR reaction in all cases. An improvement in sensitivity has been found when using Southern blotting based on probes targeted at SV40 T antigen sequences. Several researchers have used multiple primer sets to identify genomic sequences from different parts of the genome, as well as sequence-specific variation. Despite this, doubts have persisted about the authenticity of SV40 DNA detected in human tissues, and it has proved necessary to organise a multi-institutional study involving nine laboratories under the auspices of the FDA in the USA.

Accordingly, the present invention provides a new method for virus typing that is referred to hereinbelow as “Viral Evaluation using Self-Probing Amplicons” (VESPA). VESPA is a real-time PCR-based technique that uses self-probing amplicon primers, which are described by Whitcombe et al in Nat Biotechnol 17 804-7 (1999).

Such self-probing amplicon primers comprise a specific probe sequence that is held in a hairpin loop configuration by complementary stem sequences on the 5′ and 3′ sides of the probe. A fluorophore (such as 6-carboxyfluorescein) attached to the 5′-end is quenched by a moiety (such as methyl red) joined to the 3′-end of the loop. The hairpin loop is linked to the 5′-end of a primer via a PCR stopper or blocker. After extension of the primer during PCR amplification, the specific probe sequence is able to bind to its complement within the same strand of DNA. This hybridisation event opens the hairpin loop, so that fluorescence is no longer quenched and an increase in signal is observed. The PCR stopper on the primer prevents read-through to the probe, which could lead to opening of the hairpin loop in the absence of the specific target sequence. Such read-through would lead to the detection of non-specific PCR products, eg primer dimers or mis-priming events.

Accordingly, as used herein, the term “self-probing amplicon” refers to a molecule comprising a primer component, a probe component and a signalling system (which may comprise the fluorophore/quencher system as described above or an alternative), as described above and by Whitcombe (1999) (q.v.). Thus, unlike ‘molecular beacons’ and TaqMan probes that have been used, the self-probing amplicon system does not require a separate probe. The self-probing amplicons work in a unimolecular manner, leading to advantages both in terms of simplicity and signal to noise ratios when compared to the bi-molecular probing of ‘molecular beacons’ and TaqMan. Furthermore, such self-probing amplicons can be adapted to mutation or allelic discrimination by monitoring the fluorescence at a temperature where the probe has dissociated from a target with a mismatch but remains bound to a complementary target. This is different from the allelic discrimination by self-probing amplicons described by Whitcombe et al, as they used the ARMS system, whereby the primer rather than the probe is sited over the polymorphic site.

VESPA methodology is well suited to virus detection since it is simple to perform rapid, highly specific, sensitive, reproducible, and has the potential to measure viral load. The method of the invention has been tested and it has produced typing results on 108 samples, including cell lines, cervical cytobrush samples and tumour biopsies, and preliminary viral load per cell data on 16 clinically-defined samples has been obtained, as described hereinbelow in the Examples.

Furthermore, when compared to both prior art front-line virus-detection and typing methodologies, Hybrid Capture II™ and PCR-EIA, VESPA appears technically less demanding and able to produce results more rapidly. The sensitivity of VESPA in cell lines is at least two orders of magnitude better than that reported by Digene (5000 copies) for Hybrid Capture II™ (from Digene Corporation, Silver Spring, Md.), which has been approved by The Federal Drugs Administration (FDA in the USA) for HPV screening. VESPA has comparable sensitivity to other previously-published HPV detection techniques, including PCR-EIA (ie in the range of from 1 to 100 viral copies), in which the detection limit is calculated using enriched control targets against a low background of genomic DNA. The exact viral threshold for immediate risk of carcinogenesis is controversial (and may vary with HPV type and patient), but is likely to be well above VESPA's lower detection limit.

The presence of inhibitors (eg haemoglobin) that may prevent efficient DNA amplification in cervical samples has been previously reported. Using a standard chloroform extraction based method of DNA purification, we have demonstrated that it is possible to improve the signal produced by VESPA to detect HPV in cervical smear samples only weakly positive when purified by the freeze-thaw technique usually used with PCR-EIA. VESPA is type-specific and compares favourably with Hybrid Capture™, in which samples are only categorised into “Low Risk” and “High Risk” HPV types. The HPV typing achieved using VESPA correlates well in our hands with data obtained using PCR-EIA (κ=0.89), the most established HPV typing method.

In order to eliminate the false positives often associated with nested PCR, such as in the case of SV40, an assay based on highly sensitive first round amplification, in order to detect very low viral copy numbers, and highly specific second round amplification, may be used. The primer sites used in the first round of amplification may based on known and established primer sites. Preferably, for SV40, all primers and probes are capable of targetting the large T antigen site in each virus, such that each assay is equivalent. In the second step, using self-probing amplicon primers and real-time PCR, amplification and detection take place concurrently.

As well as detection and typing, VESPA has the potential to estimate viral load (ie per cell rather than gross amount of virus present). There is increasing evidence that viral load per cell is a critical determinant in patient prognosis. Indeed, the so-called “high risk” types may not be more potent due to the increased oncogenicity of their transforming proteins, for example, but simply because they proliferate more efficiently, overwhelming the immune response.

In order to expand the capability of VESPA for use in virus screening, the present invention further provides a degenerate virus self-probing amplicon mix for use in conjunction with a tailed general primer. By using a tailed primer, it is possible to introduce a consensus site that enables a single self-probing amplicon to recognise many different virus amplification products. Theoretically, this primer combination can detect over forty different HPV types.

In the PCR-EIA technique described by Jacobs et al in J Clin Microbiol 35, 791-5 (1997), viral DNA is amplified using the prior art consensus primers (GP5+/GP6+), then analysed by ethidium bromide staining of electrophoresed agarose gels. If an amplification product is observed, then it is typed by ELISA using type-specific probes. In practice, however, all samples are typed by ELISA, since agarose gel electrophoresis is not sensitive enough to detect poorly amplified DNA. VESPA could circumvent this problem by providing improved sensitivity at the pre-typing stage. Although the concept of a tailed primer is known, the use of the tail as a primer site and the primer as a probe-binding site for a self-probing amplicon is new.

Therefore, the method of the invention for the characterisation of viruses such as human papillomavirus or SV40 infection is quicker (<1 hour); more specific (single base discrimination); and less laborious (single step) than currently available techniques and, unlike most techniques, is capable of estimating viral load per cell. It can also be used to determine integration status of the virus. Especially important is the ability of the technique of the invention to determine a plurality of virus types by using tailed primers.

The invention is further described below, with reference to the following Figures:

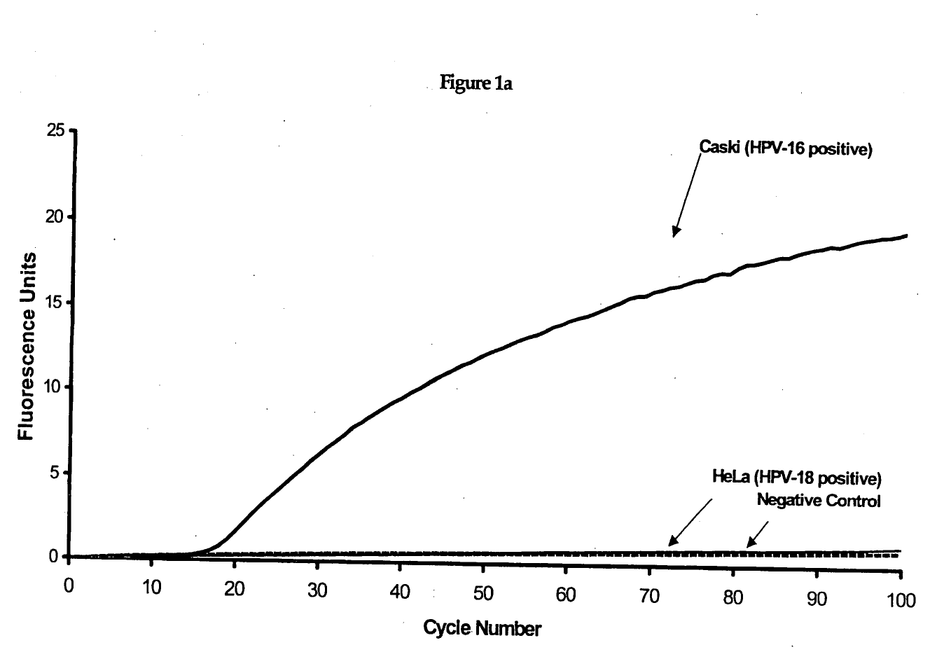

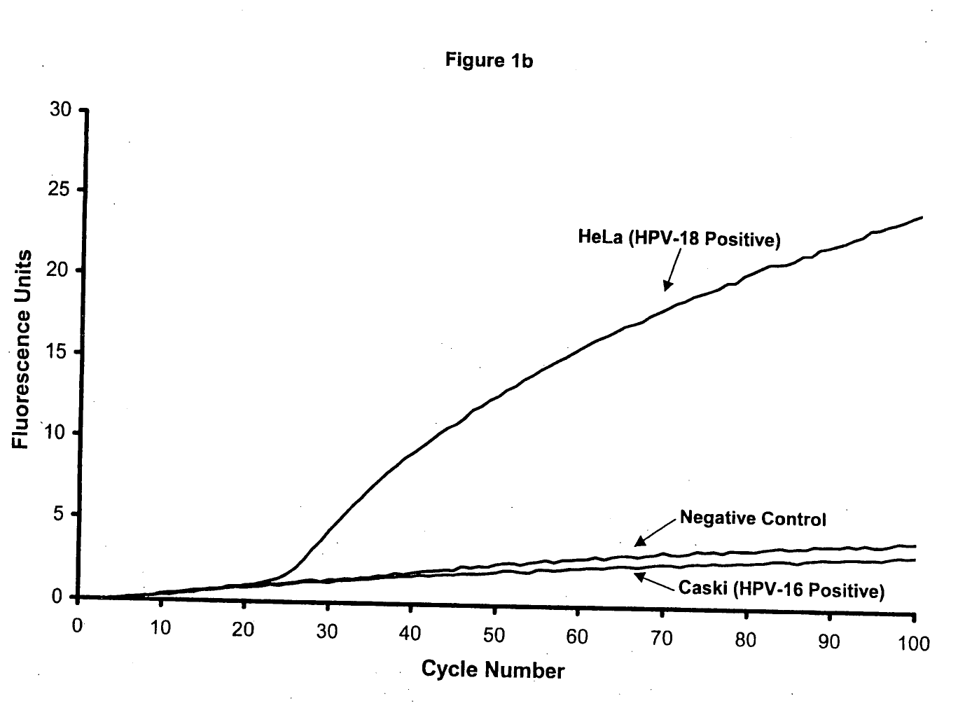

FIG. 1: relates to HPV-16 detection by the method of the invention, in which:

FIG. 1a shows the results of HPV typing reactions using self-probing amplicon primers specific for HPV-16 (Sc16), a positive control (HeLa for HPV-18); a negative control (no DNA); and DNA extracted from a HPV 16 specific cell line (Caski); and

FIG. 1b shows the results of HPV typing reactions using the self-probing amplicon primers specific for HPV-18 (Sc18); a positive control (Caski for HPV-16), a negative control (no DNA); and DNA extracted from an HPV-18 containing cell line (HeLa).

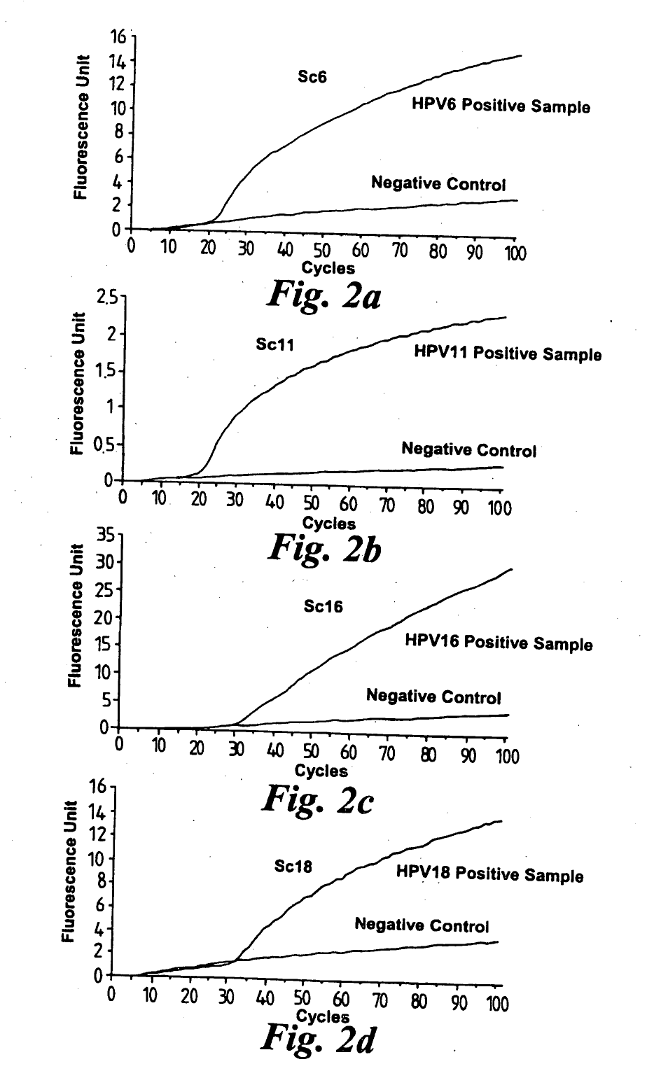

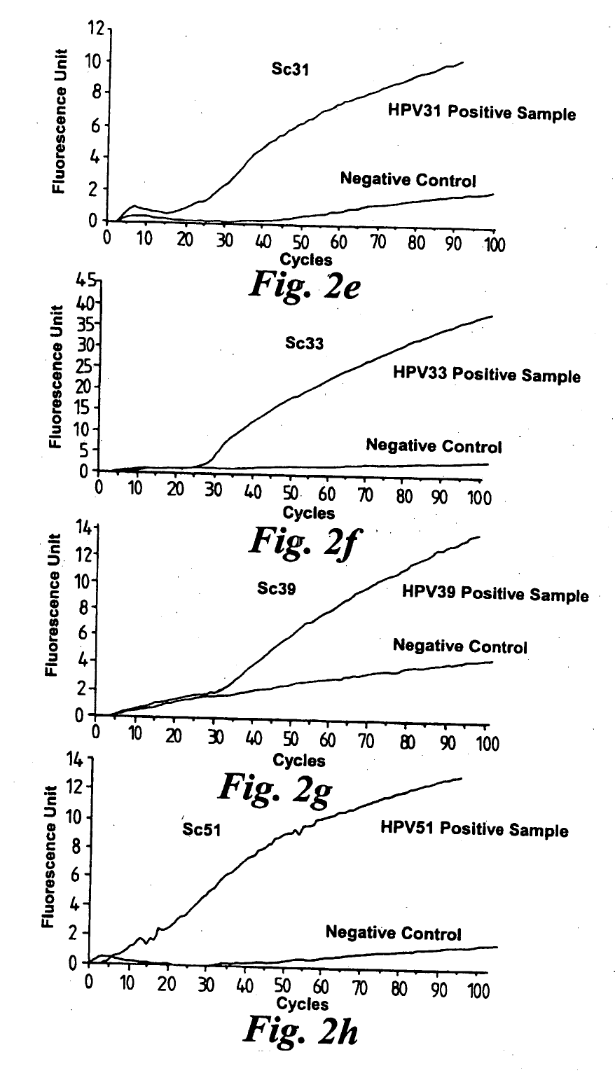

FIG. 2 relates to examples of positive traces produced by VESPA and shows the results of HPV typing experiments using clinical samples previously typed using PCR-EIA. Primers specific for HPV-6 (Sc6) are shown in FIG. 2a, HPV-11 (Sc11) in FIG. 2b, HPV-16 (Sc16) in 2c, HPV-18 (Sc18) in 2d, HPV-31 (Sc31) in 2e, HPV-33 (Sc33) in 2f, HPV-39 (Sc39) in 2g, and HPV-51 (Sc51) in 2h.

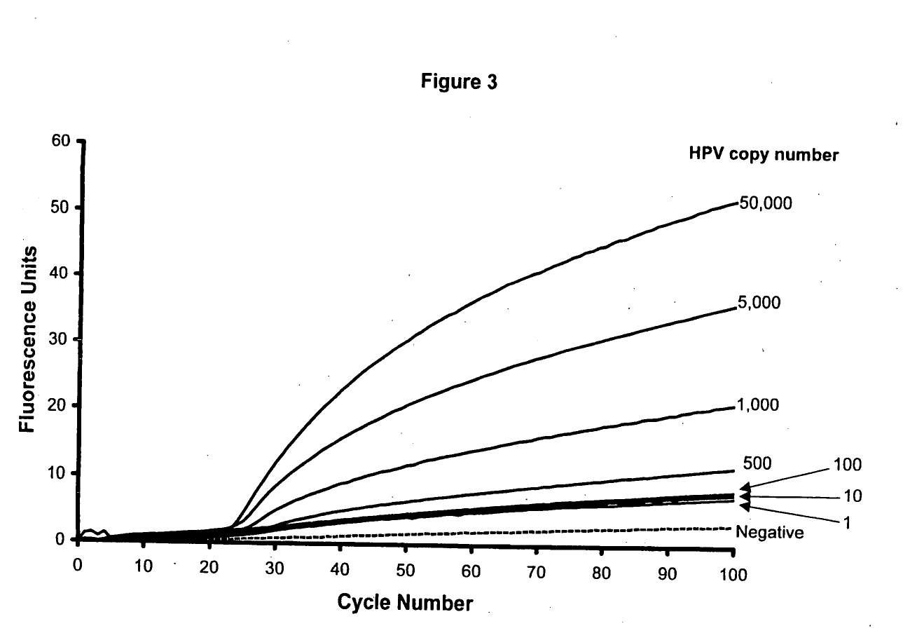

FIG. 3 demonstrates the quantitative nature of VESPA for HPV-16. It shows an HPV-16 dilution series using Sc16. A dilution series of SiHa cells was made from 50,000 cells per reaction to 1 cell per reaction.

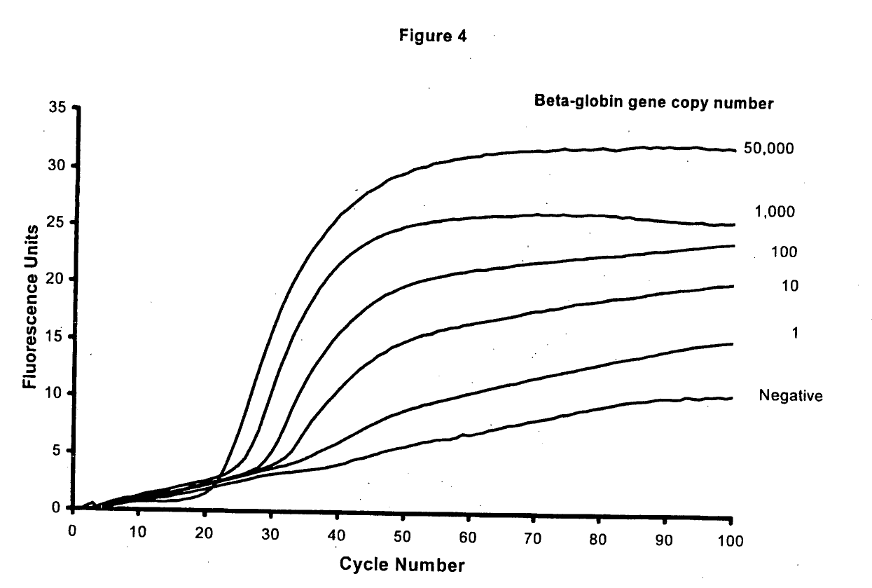

FIG. 4 demonstrates the quantitative nature of VESPA for human beta-globin using ScBG and the dilution series as for FIG. 3.

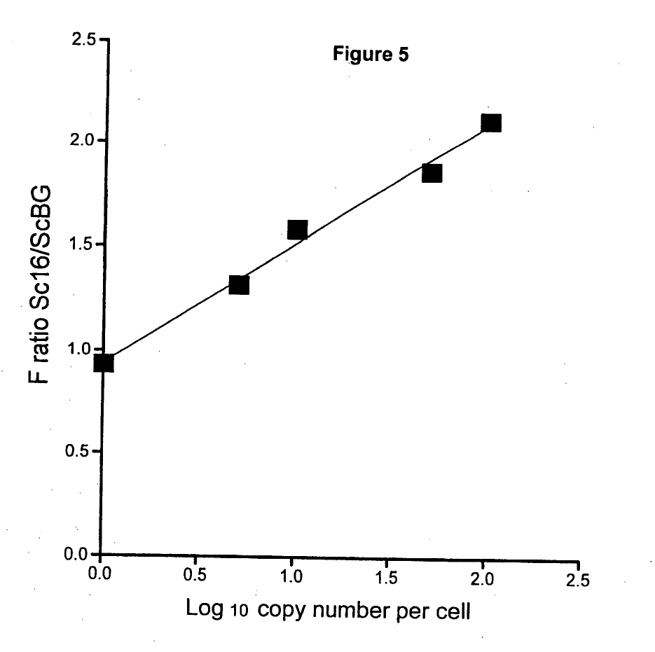

FIG. 5 is a graph showing estimation of viral load using VESPA.

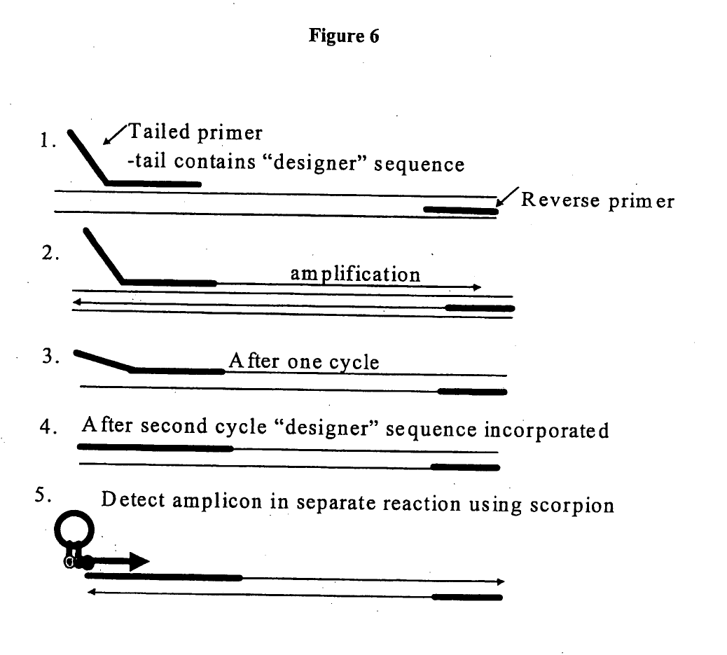

FIG. 6 is a diagrammatic representation of a strategy for virus, such as degenerate HPV, detection using VESPA.

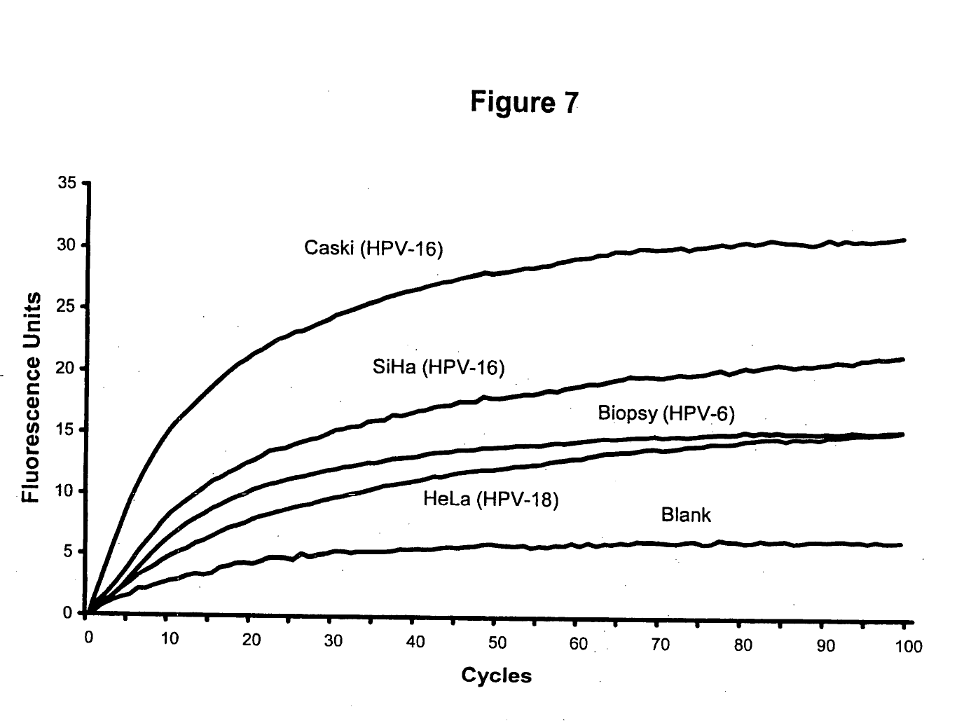

FIG. 7 demonstrates the ability of the degenerate self-probing amplicon mix to detect HPV types-6, -16 and -18.

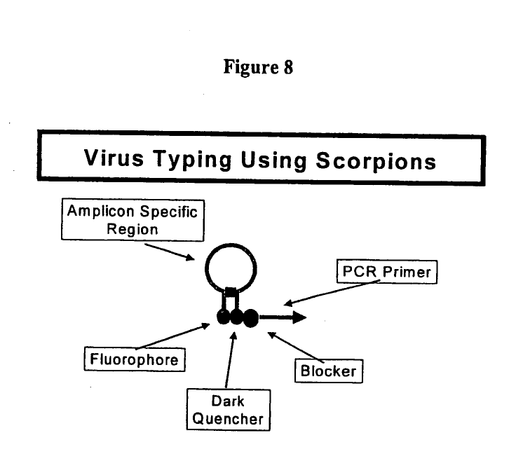

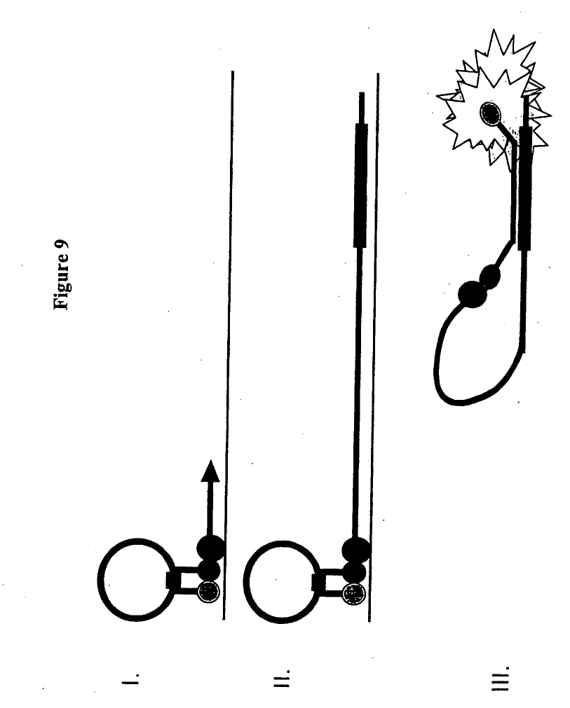

FIGS. 8 and 9 are schematic representations of virus, such as HPV, typing using self-probing amplicons, in which FIG. 8 is a diagram of a self-probing amplicon and FIG. 9 shows the extension and binding stages of the self-probing amplicon with respect to the viral DNA.

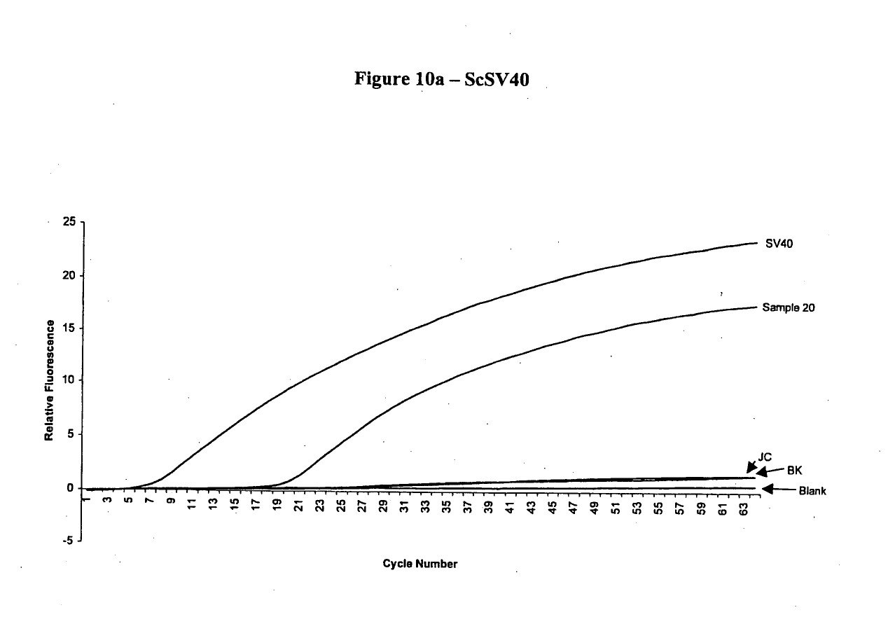

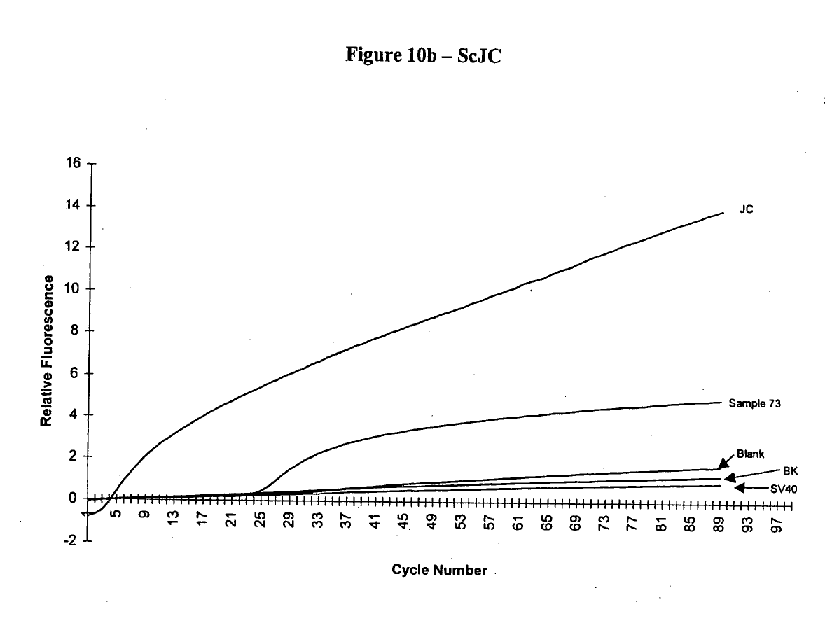

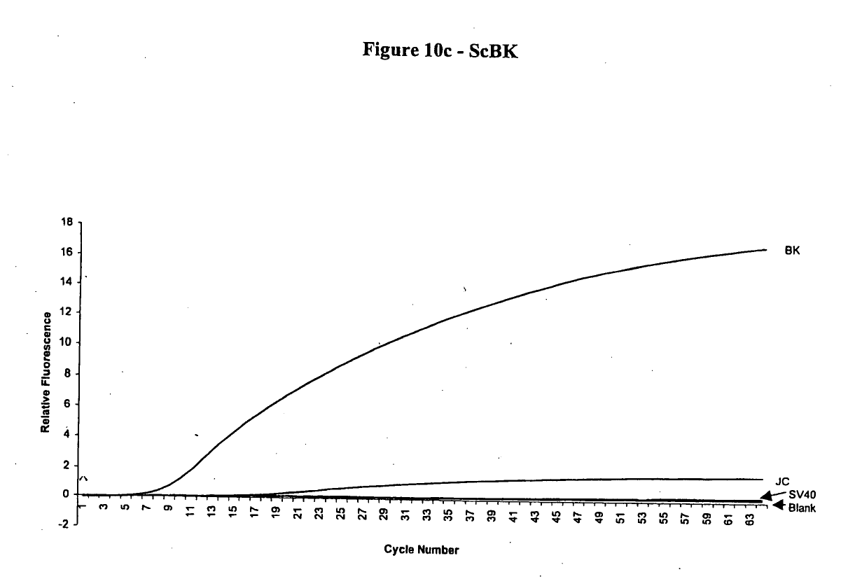

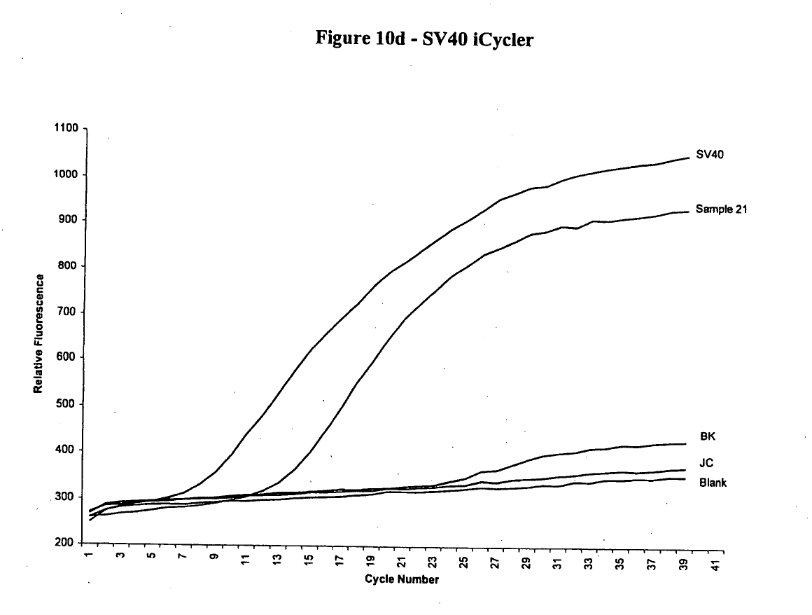

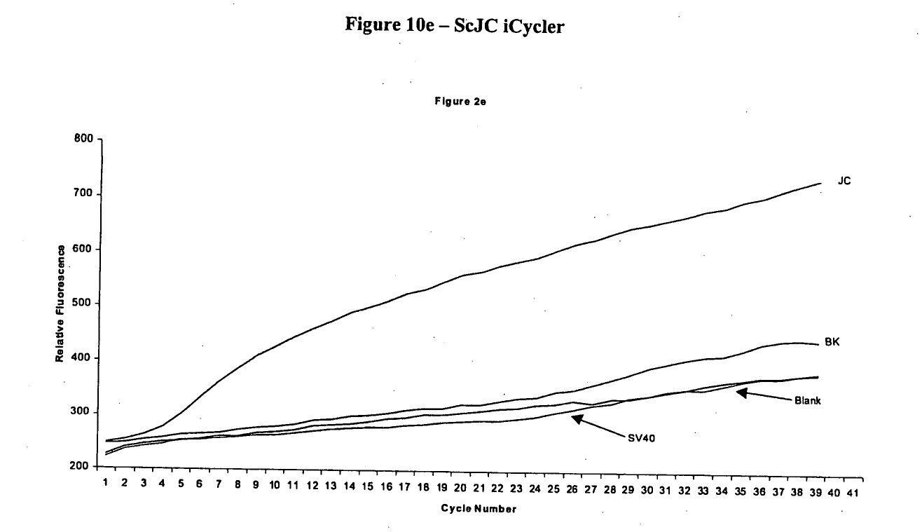

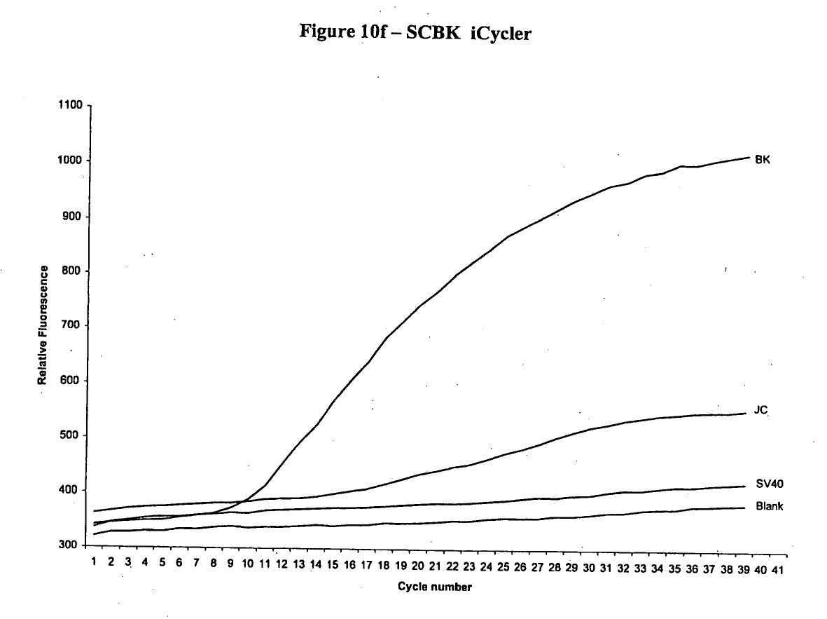

FIG. 10 relates to results obtained in applying the VESPA technique to SV40. Shown in FIG. 10a-c are LightCycler fluorescence profiles produced by ScSV40, ScJC and ScBK, respectively. Each self-probing amplicon (Sc) is shown challenged with pre-amplified Ori-3 (SV40), Mad 1 (JC), X (BK) and sample DNA. Shown in FIG. 10d-f are iCycler fluorescence profiles produced under the same conditions.

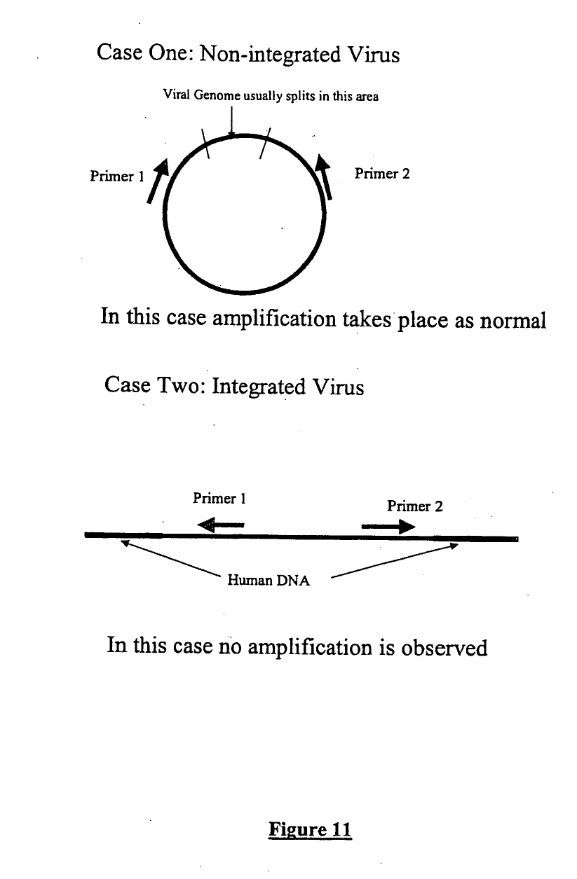

FIG. 11 is a schematic representation of a strategy for determining integration state of ca virus, using the method according to the invention.

The present invention, therefore, provides a method for one or more of:

(a) detection;

(b) typing;

(c) determination of viral load per cell; and/or

(d) determination of the integration state of an animal, including a mammalian, virus in a sample from an animal, including a mammal, suspected of comprising one or more target viral nucleic acid sequence(s),

which method comprises:

(IA) contacting the sample with a self-probing amplicon (‘virus self-probing amplicon’) comprising
(i) a virus primer capable of hybridising to at least one target viral nucleic acid sequence and undergoing amplification thereof under primer amplification conditions to form a virus primer extension product;
(ii) a virus probe comprising a nucleic acid sequence complementary to a target sequence of the virus primer extension product and capable of hybridisation thereto, provided that the self-probing amplicon is adapted to ensure that the virus probe is unresponsive to amplification under the primer amplification conditions; and
(iii) a member of a virus signalling system, which system is capable of causing a detectable signal to be effected on hybridisation of the virus probe sequence to the virus primer extension product, whereby presence or absence of the target viral nucleic acid sequence in the sample is indicated by the detectable signal;

(IB) amplifying the product of step (IA) under the primer amplification conditions to an extent enabling the detectable signal to be effected after step (II); and

(II) separating the virus primer extension product from the target viral nucleic acid sequence; allowing the virus probe to hybridise to the target sequence of the virus primer extension product; and monitoring the signalling system.

(FIGS. 8 and 9 illustrate the method, schematically.)

Particularly preferred is a method according to this invention, wherein the nucleic acid sequence(s) is/are DNA sequence(s).

In order to be particularly effective in determining viral load per cell, the method is preferably carried out using a self-probing amplicon designed to detect DNA of a cell housekeeping gene. In the context of this invention, “cell housekeeping” or “housekeeping” gene refers to a gene that is stably present in the cell and therefore suitable for acting as a baseline indicator for the presence of the cell in the sample.

Accordingly, the present invention further provides a method for one or more of:

(a) detection;

(b) typing;

(c) determination of viral load per cell; and/or

(d) determination of the integration state of a virus in a sample suspected of comprising one or more target viral nucleic acid sequence(s),

which method comprises:

(IA) contacting the sample with a self-probing amplicon (‘virus self-probing amplicon’) comprising
(i) a virus primer capable of hybridising to at least one target viral nucleic acid sequence and undergoing amplification thereof under primer amplification conditions to form a virus primer extension product;
(ii) a virus probe comprising a nucleic acid sequence complementary to a target sequence of the virus primer extension product and capable of hybridisation thereto, provided that the self-probing amplicon is adapted to ensure that the virus probe is unresponsive to amplification under the primer amplification conditions; and
(iii) a member of a virus signalling system, which system is capable of causing a detectable signal to be effected on hybridisation of the virus probe sequence to the virus primer extension product, whereby presence or absence of the target viral nucleic acid sequence in the sample is indicated by the detectable signal; and

(IB) amplifying the product of step (IA) under the primer amplification conditions to an extent enabling the detectable signal to be effected after step (II);

(II) separating the virus primer extension product from the target viral nucleic acid sequence; allowing the probe to hybridise to the target sequence of the virus primer extension product; and monitoring the signalling system;

(IIIA) contacting a housekeeping nucleic acid sequence from the sample with a self-probing amplicon (‘housekeeping self-probing amplicon’) comprising
(i) a housekeeping primer capable of hybridising to the housekeeping nucleic acid sequence and undergoing amplification thereof under primer amplification conditions to form a housekeeping primer extension product;
(ii) a housekeeping probe comprising a nucleic acid sequence complementary to a target sequence of the housekeeping primer extension product and capable of hybridisation thereto, provided that the housekeeping self-probing amplicon is adapted to ensure that the probe is unresponsive to amplification under the primer amplification conditions; and
(iii) a member of a housekeeping signalling system, which system is capable of causing a detectable signal to be effected on hybridisation of the housekeeping probe sequence to the housekeeping primer extension product, whereby presence or absence of the target housekeeping nucleic acid sequence in the sample is indicated by the detectable signal;

(IIIB) amplifying the product of step (IIIA) under the primer amplification conditions to an extent enabling the detectable signal to be effected after step (IV); and

(IV) separating the housekeeping primer extension product from the housekeeping nucleic acid sequence; allowing the housekeeping probe to hybridise to the target sequence of the housekeeping primer extension product; and monitoring the housekeeping signalling system.

Suitable housekeeping genes include β-globin, actin, tropomyosin and glyceraldehyde phosphate dehydrogenase (GAPDH). Preferably, the housekeeping gene is β-globin, which is particularly suitable for acting as a human genomic DNA reference gene in the viral load per cell analysis according to this invention.

Suitably, viral load per cell can be determined by comparing the signals effected on hybridisation of, on one hand, the ‘viral self-probing amplicon’ and, on the other hand, the ‘housekeeping self-probing amplicon’. For example, viral load per cell can be quantified as a simple ratio of the two signals. (FIGS. 4 and 5 refer.)

Conveniently, for determining both viral type(s) and load per cell in the same experiment, a plurality of signalling systems may be employed, whereby the signals distinguishably identify presence of virus type and housekeeping gene, respectively. Otherwise, the steps (I) plus (II) and (III) plus (IV), respectively, may be carried out in separate experiments.

In the methods of this invention, suitable signalling systems include fluorescence-based systems, such as wherein the self-probing amplicon further comprises a fluorophore/quencher pair, such as 6-carboxyfluorescein/methyl red. Nevertheless, other signalling systems may be employed.

Preferably, amplification step(s) are carried out using the polymerase chain reaction (PCR), although the method(s) may be adapted to use alternative amplification methodology. More preferably, PCR is carried out in ‘real time’. Accordingly, amplicon detection is preferably carried out using real-time PCR machines, especially the iCycler (available from Bio-Rad Laboratories, UK).

The self-probing amplicon may be adapted in any suitable way known in the art to ensure that the probe is unresponsive to amplification under the primer amplification conditions. For example, to prevent read-through to the probe component of the self-probing amplicon, the primer component preferably further comprises an amplification blocker or stopper, such as hexethyl glycol (HEG).

Separation of the primer extension product from the target nucleic acid sequence can be undertaken by standard methods in the art. Preferably, the separation steps (II) and/or (IV) are carried out by heat denaturation.

The target virus nucleic acid sequence is preferably one that is capable of indicating the presence of a virus that is associated with a disease or clinical condition in an animal, especially a mammal, more especially man. Particularly preferred is when the virus is human HPV. Preferably, the virus is selected from one or more of HPV types 6, 11, 16, 18, 31, 33, 39, 40, 42, 43, 44, 45, 51, 52, 56, 58, 59, 66 and 68.

In such cases, the self-probing amplicon is suitably one having a sequence selected from SEQ ID NOs 1 to 9, corresponding to self-probing virus amplicons named Sc6, 11, 16, 18, 31, 33, 39, 51 and Sc56, respectively, where Scx refers to the amplicon for self-probing HPV type x (refer to Table 2 in Example 1 hereinbelow). Especially preferred is when the probe is the probe component of the above-noted sequences, namely a sequence selected from SEQ ID Nos [21] to [29]. (FIGS. 1 and 3 refer.)

| | |
|---|---|
| 21 | ATAAAGAGTACATGCGT |
| 22 | CAGATTATAAGGAATACATGC |
| 23 | AGTACCTACGACATGGG |
| 24 | AGCAGTATAGCAGACATG |
| 25 | GAGTATTTAAGAGATGGTG |
| 26 | CTTTATGCACACAAGAAC |
| 27 | AATATACCAGGCACGTG |
| 28 | GCAATATATTAGGCATGGG |
| 29 | TCAGTACCTTAGACATGTG |

Preferably, the primer is the primer component of the above-noted sequences, namely, a sequence selected from GP6+ and SEQ ID Nos: 32-40.

| | |
|---|---|
| GAAAAATAAATTGTAAATCATACTC | SEQ ID NO:32 |
| GAAAAATAAACTGTAAATCAAACTC | SEQ ID NO:33 |
| GAAAAATAAACTGTAAATCATATTC | SEQ ID NO:34 |
| GAAAAATAAACTGCAAATCATATTC | SEQ ID NO:35 |
| GAAATATAAATTGTAAATCAAATTC | SEQ ID NO:36 |
| GAAAAACAAACTGTAGATCATATTC | SEQ ID NO:37 |
| GAAATATAAATTGTAAATCATACTC | SEQ ID NO:38 |
| AAAAATAAATTGCAATTCATACTC | SEQ ID NO:39 |
| GAAAAACAAATTGTAACCCATATTC | SEQ ID NO:40 |

Suitably, the virus may be SV40, or the highly prevalent and homologous JC and BK viruses. In such a case, the self-probing amplicon is preferably one having a sequence selected from those listed in Table 8 of Example 6 hereinbelow, where Scx refers to the amplicon for self-probing SV40, JC or BK viruses (x). These sequences specifically target sites known to be capable of reliably detecting polyomaviruses and to be directly comparable with results obtained by other methods in this art. Preferred forward primers are the P1 primers, as indicated in Table 8.

In the method of this invention, particularly as applied to HPV (VESPA-HPV), PCR amplification is preferably carried out using the GP5+ reverse primer, as defined herein. GP5+ is a known degenerate primer capable of amplifying the 20 most common types of HPV; however, other degenerate primers could be used instead. Particularly suitable for carrying out VESPA-SV40 is the use of P2 or P3 as reverse primers, as detailed in Table 8. A preferred such method is wherein one or more of the amplification step(s) is/are carried out using ‘nested’ PCR

A preferred self-probing amplicon for use in viral load (per cell) determination is referred to in Table 3 in Example 2 hereinbelow as ScBG [SEQ ID No: 11]. Especially preferred is when the probe component of such a self-probing amplicon is SEQ ID NO: [31]:

| | |
|---|---|
| 31 | ATGGTGTCTGTTTGAG |

More preferably still, the ‘viral self-probing amplicons’ include those that allow detection of changes in the E1, E2, E6 and/or E7 HPV genes in order to determine integration state of the viral genome in the sample cell genome. Integration status can be measured using two different assessment methods:
(i) Ratio of circular viral DNA to linear viral DNA; and
(ii) Ratio of cell cycle control viral proteins to cell transforming proteins.

The first of these methods (i) is illustrated in FIG. 11 (as described by Park et al in Gynecol Oncol 65(1) 121-9 (1997)). Each sample is assessed, using the method of the invention, for the quantitative amount of viral DNA in circular form. This assessment may be achieved by using self-probing amplicons designed to contact the viral DNA before the putative viral DNA break point (in the middle of the E1 protein) and a reverse primer after the putative viral break point (in the E6 or, preferably, E7 protein). This assessment reflects the quantitative amount of viral DNA in circular form. This result may then be compared with total viral DNA, as determined by the method of the invention described above, to give a percentage of viral DNA in circular form.

The second of these methods (ii) relies on the fact that, upon integration of viral DNA into the host genome, the section of viral DNA responsible for the control of viral DNA replication and translation into proteins is excised. This allows the proteins known to be responsible for carcinogenesis to replicate out of control. Thus, by quantitatively measuring the ratio of the control proteins (E1 and E2) to the transforming proteins (E6 and E7), it is possible to assess the likelihood that the sample is derived from a patient that is about to undergo malignant transformation.

Therefore, the invention further provides a method for one or more of:

(a) detection;

(b) typing;

(c) determination of viral load per cell; and/or

(d) determination of the integration state of a virus in a sample suspected of comprising one or more target viral nucleic acid sequence(s),

which method comprises:

(IA) contacting the sample with a self-probing amplicon (‘virus self-probing amplicon’) comprising
(i) a virus primer capable of hybridising to at least one target viral nucleic acid sequence and undergoing amplification thereof under primer amplification conditions to form a virus primer extension product;
(ii) a virus probe comprising a nucleic acid sequence complementary to a target sequence of the virus primer extension product and capable of hybridisation thereto, provided that the self-probing amplicon is adapted to ensure that the virus probe is unresponsive to amplification under the primer amplification conditions; and
(iii) a member of a virus signalling system, which system is capable of causing a detectable signal to be effected on hybridisation of the virus probe sequence to the virus primer extension product, whereby presence or absence of the target viral nucleic acid sequence in the sample is indicated by the detectable signal; and

(IB) amplifying the product of step (IA) under the primer amplification conditions to an extent enabling the detectable signal to be effected after step (II);

(II) separating the virus primer extension product from the target viral nucleic acid sequence; allowing the virus probe to hybridise to the target sequence of the virus primer extension product; and monitoring the signalling system,

wherein the ‘viral self-probing amplicon(s)’ is/are adapted to allow detection, quantification or assessment of the E1, E2, E6 and/or E7 HPV genes.

Accordingly, the present invention still further provides such ‘viral self-probing amplicons’ as are defined hereinbelow in Table 6, Example 4, namely: Sc16-E1mid, Sc16-E2, Sc16-E6, Sc18-E1mid, Sc18-E2 and Sc18-E6 [SEQ ID Nos: 11 to 17, respectively]. The present invention further provides sequences comprising the probe part of each of these sequences and the novel primer parts of these sequences, respectively SEQ ID NOs 41 to 46 and 47 to 52.

| | | |
|---|---|---|
| Sc16-E1mid | GCAAAGAGTAATCATTA | SEQ ID NO:41 |
| probe | | |
| Sc16-E2 | TTGTCATATAGACATATCATTTTCAT | SEQ ID NO:42 |
| probe | | |
| Sc16 E6 | CGAATGTCTACATATCATGGC | SEQ ID NO:43 |
| probe | | |
| Sc18 E1mid | TCGGTGTCTCCATGTTG | SEQ ID NO:44 |
| probe | | |
| Sc18-E2 | TACATTGTCATGGTCTATGAT | SEQ ID NO:45 |
| probe | | |
| Sc18-E6 | CTGGAATGCTATATCATG | SEQ ID NO:47 |
| probe | | |
| Sc16-E1mid | CAGAATGGATACAAAGACAAACAGT | SEQ ID NO:47 |
| primer | | |
| Sc16-E2 | CAACGTTTAAATGTGTGTCAGGA | SEQ ID NO:48 |
| primer | | |
| Sc16 E6 | AAGTTACCACAGTTATGCACAGAGC | SEQ ID NO:49 |
| primer | | |
| Sc18 E1mid | AGTAATGGGAGACACACCTGAGT | SEQ ID NO:50 |
| primer | | |
| Sc18-E2 | GCAGACACCGAAGGAAACCC | SEQ ID NO:51 |
| primer | | |
| Sc18-E6 | ACCCAGAAAGTTACCACAGTTAT | SEQ ID NO:52 |
| primer | | |

Conveniently, to avoid multiple testing of a sample using many different ‘virus self-probing amplicons’, is the method of the invention wherein the target comprises more than one nucleic acid sequence from more than one virus and/or the virus primer component exhibits some degeneracy with respect to the target, whereby the virus primer is not entirely complementary to each one of the nucleic acid sequences of the target.

However, for screening purposes, it may be more convenient to carry out a preliminary assay to establish first whether there is any of a virus or category of virus present in the sample and, if this assay were to prove positive, then to proceed to the specific typing and quantitation assays enabled by the method of this invention. Such a preliminary assay may comprise any previously known method for, virus detection, including those mentioned hereinbefore. Preferably, however, the preliminary assay comprises the method of the invention wherein a tailed primer is first incorporated into the viral primer extension product. The tail comprises a nucleic acid sequence capable of amplifying, eg under PCR conditions, the viral nucleic acid sequence of a plurality of viruses or virus, such as HPV, types. It is believed that this is the first time the concept of tailed primers has been applied to self-probing amplicons.

Accordingly, the present invention further provides a method comprising the following steps:

(0)(A) contacting a target viral nucleic acid sequence from the sample with ‘tailed primer’, which comprises:
(i) a primer region comprising a nucleic acid sequence (‘consensus primer sequence’) complementary to a consensus sequence of the viral nucleic acid sequence and capable of hybridisation thereto and undergoing amplification thereof under primer amplification conditions to form a tailed primer extension product; and
(ii) a tail region comprising a unique sequence not present in or prepared by any component of this method (‘designer’ sequence); and

(0)(B) carrying out at least two rounds of amplification under the primer amplification conditions, whereby the ‘designer’ sequence becomes incorporated into the primer extension product; and

wherein the primer component of the ‘virus self-probing amplicon’ is capable of binding to the ‘designer’ sequence and the probe component of the virus self-probing amplicon is complementary to the consensus primer sequence.

Accordingly, in a preferred method of the invention in order to test for any HPV type in a single reaction, a unique self-probing amplicon target site is introduced into the amplification (primer extension) product. By attaching a unique ‘designer tail’ to the prior art consensus primers (such as one capable of amplifying all 20 of the common HPV types), this aim can be achieved. A preferred tailed primer for use in the present invention is shown in Table 7 of Example 5 [SEQ ID NO: 18]. Two suitable degenerate self-probing amplicons for use in the present invention are shown in Table 7 of Example 5 [SEQ ID NOs: 19 & 20]; these sequences also comprise preferred primer and probe components. (FIGS. 6 and 7 refer.)

Accordingly, the present invention further provides the novel tail part [SEQ ID NO: 10] of these sequences; the probe part of these sequences being designed to bind to the known GP6+ sequence.

| | | |
|---|---|---|
| ATGTGGAAACATGCATGG | GP6+ tail comprised | SEQ ID NO:10 |
| | in [SEQ ID NO:18] | |

Especially preferred is when the method of the invention is incorporated into a screening programme and the self-probing amplicons of this invention are for use therein.

Accordingly, the invention still further provides:

(a) A screening method for screening an individual suspected of a viral infection, which screening method comprises:
(i) obtaining a sample of a nucleic acid sequence from the individual; and
(ii) carrying out, on the sample, a method according to the invention as described above, whereby presence of the detectable signal from the virus signalling system indicates presence of the viral infection and absence of the detectable signal indicates absence of the viral infection;

(b) A screening method according to (a), which screening method further indicates the presence or absence of specific viral type(s);

(c) A screening method according to (a) or (b), which screening method further indicates viral load per cell, when virus is present (being zero when virus is absent);

(d) A screening method according to any of (a) to (c), which screening method further indicates integration status of the virus, when present;

(e) A screening method according to any of (a) to (d), which screening method is adapted for screening for cervical cancer, recurrent respiratory papillomatosis or any other condition associated with the presence in the individual of a human papillomavirus (HPV); and

(f) A screening method according to any of (a) to (e), which screening method is adapted for screening for one or more of: mesotheliomas, including cancers of the chest and lung; osteosarcomas; pituitary, thyroid, brain and neurological tumours (eg glioblastomas, astrocytomas, ependymomas and papillomas of the choroids plexus); and other conditions associates with SV40, JK and/or BK virus(es).

The methods of the invention, although preferably carried out on DNA, can be adapted by methods known to those skilled in the art to be carried out on RNA. In this case, any commercially available reverse transcriptase could be employed first to transcribe the RNA to DNA, and then the method carried out as described above.

The present invention also provides a diagnostic kit for use in a method of the invention, which kit comprises one or more of the virus self-probing amplicons, housekeeping self-probing amplicons or tailed primers of the invention. Particularly preferred is a kit suitable for use in a method according to the invention for determining viral load per cell, and accordingly such comprises at least two, and suitably four or more, self-probing amplicons, including at least one housekeeping self-probing amplicon and, optionally, a tailed primer. Also preferred is a kit comprising at least one self-probing amplicon for targeting at least one of the E1, E2, E6 and E7 HPV genes.

Many of these self-probing amplicons are themselves new and inventive. Accordingly, the invention further provides:

(a) a novel self-probing amplicon as described herein, including:
(i) SEQ IDs NOs: 1-9 (Sc 6, 11, 16, 18, 31, 33, 39, 51 & 56)
(ii) SEQ ID NO: 11 (ScBG)
(iii) SEQ ID NOs: 12-17 (Sc16-E1mid, Sc16-E2, Sc16 E6, Sc18 E1 mid, Sc18-E2, Sc18-E6)
(iv) SEQ ID NOs: 18-20 (tailed primers)
(v) Sequences specified in Table 8 hereinbelow (self-probing amplicons for use in SV40, JC or BK determination)

(b) a novel probe component of (a), including:
(i) SEQ IDs NOs: 21-29 (probe components of (Sc 6, 11, 16, 18, 31, 33, 39, 51 & 56)
(ii) SEQ ID NO: 31 (probe component of ScBG)

(iii) SEQ IDS NOs: 41-46

| | | |
|---|---|---|
| Sc16-E1mid | GCAAAGAGTAATCATTA | SEQ ID NO:41 |
| probe | | |
| Sc16-E2 | TTGTCATATAGACATATCATTTTCAT | SEQ ID NO:42 |
| probe | | |
| Sc16 E6 | CGAATGTCTACATATCATGGC | SEQ ID NO:43 |
| probe | | |
| Sc18 E1mid | TCGGTGTCTCCATGTTG | SEQ ID NO:44 |
| probe | | |
| Sc18-E2 | TACATTGTCATGGTCTATGAT | SEQ ID NO:45 |
| probe | | |
| Sc18-E6 | CTGGAATGCTATATCATG | SEQ ID NO:46 |
| probe | | |

(iv) SEQ IDS NOs: 59 and 60, being the probe component of SEQ ID NOs: 19-20 (tailed primers)

| | |
|---|---|
| GAAGAATATGATTTACA | SEQ ID NO:59 |
| GAGGAATATGATTTACA | SEQ ID NO:60 |

(v) SEQ ID Nos: 53 to 55, being the probe component of the sequences listed in Table 8 (SV40, BK and JC self-probing amplicons);

| | |
|---|---|
| ACCCCAAGGACTTTCCT | SEQ ID NO:53 |
| CCTATGGAACAGATGAATG | SEQ ID NO:54 |
| ACCCTAAAGACTTTCCC | SEQ ID NO:55 |

(c) a novel primer component of (a), including:

(i) SEQ IDS NOs: 3240 (primer components of Sc 6, 11, 18, 31, 33, 39, 51 & 56), respectively:

| | |
|---|---|
| GAAAAATAAATTGTAAATCATACTC | SEQ ID NO:32 |
| GAAAAATAAACTGTAAATCAAACTC | SEQ ID NO:33 |
| GAAAAATAAACTGTAAATCATATTC | SEQ ID NO:34 |
| GAAAAATAAACTGCAAATCATATTC | SEQ ID NO:35 |
| GAAATATAAATTGTAAATCAAATTC | SEQ ID NO:36 |
| GAAAAACAAACTGTAGATCATATTC | SEQ ID NO:37 |
| GAAATATAAATTGTAAATCATACTC | SEQ ID NO:38 |
| AAAAATAAATTGCAATTCATACTC | SEQ ID NO:39 |
| GAAAAACAAATTGTAACCCATATTC | SEQ ID NO:40 |

(iii) SEQ IDS Nos: 47-52, in particular, the E1 mid primer sequences [SEQ ID Nos: 47 and 50]:

| | | |
|---|---|---|
| Sc16-E1mid | CAGAATGGATACAAAGACAAACAGT | SEQ ID NO:47 |
| primer | | |
| Sc16-E2 | CAACGTTTAAATGTGTGTCAGGA- | SEQ ID NO:48 |
| primer | | |
| Sc16 E6 | AAGTTACCACAGTTATGCACAGAGC | SEQ ID NO:49 |
| primer | | |
| Sc18 E1mid | AGTAATGGGAGACACACCTGAGT | SEQ ID NO:50 |
| primer | | |
| Sc18-E2 | GCAGACACCGAAGGAAACCC | SEQ ID NO:51 |
| primer | | |
| Sc18-E6 | ACCCAGAAAGTTACCACAGTTAT | SEQ ID NO:52 |
| primer | | |

(iv) SEQ IDS NO: 61 being the primer component of SEQ ID NOs: 19-20 (tailed primers)

SEQ ID NO: 61 GTGGAAACATGCATGGCGAC

(v) SEQ ID Nos: 56 to 58, being the primer component of the sequences listed in Table 8 (SV40, BK and JC self-probing amplicons);

SEQ ID NO: 56 AGCATGACTCAAAAAACTTAGCAATTCT
SEQ ID NO: 57 TTCTCATTAAATGTATTCCACCAGGATT
SEQ ID NO: 58 AGCTTGACTAAGAAAGTGGTGTAGATCA

(d) [SEQ ID NO: 10] ATGTGGAAACATGCATGG tail sequence of GP6+ tailed primer

Furthermore, the invention provides the use of such self-probing amplicons, including the probe and primer components thereof, in a method or in the preparation of a kit as hereinbefore described.

The present invention will now be illustrated by the following Examples.

### EXAMPLE 1

### Viral Detection Using VESPA

General Materials and Methods

Cell Lines

The HeLa, Caski and SiHa cell lines were provided by Dr Steve Man, University of Wales College of Medicine, Cardiff, UK, and are available from the ATCC (American Type Culture Collection, http://www.atcc.org/)

Clinical Samples

Patients were either recruited during routine colposcopy clinics at Llandough Hospital, Cardiff, UK, or as part of an MRC field study in West Africa. Informed consent was obtained from all subjects who were asked for permission to assess their smear for HPV, but the methodology remained confidential. Cervical samples were collected using conical cytobrushes, and transported in 0.5 ml of Digene transport medium (from Silver Spring, Md., USA). Samples taken in the UK were stored at 4° C. for up to 24 hours before processing. Samples collected in the Gambia were stored, frozen in liquid nitrogen, and processed within one month. Biopsies were collected, with informed consent, from patients undergoing treatment for Recurrent Respiratory Papillomatosis and cervical cancer at The University Hospital of Wales, Cardiff, UK and Llandough Hospital, Penarth, UK.

DNA Purification

DNA was purified from cell lines by re-suspension of cells in 640 μl of Nuclear Lysis Buffer (10 mM Tris HCl, 0.4M NaCl, 2 mM ethylenediamine tetra-acetate pH 8.0, 10% sodium dodecyl sulphate), 100 μl of 6M NaCl and 740 μl of chloroform. The solution was thoroughly mixed, centrifuged and the top phase extracted. DNA was precipitated by the addition of 1 ml 95% ethanol and pelleted by centrifugation. The pellet was washed twice with 70% ethanol, dried in a rotary evaporator and re-suspended in 500 μl of de-ionised water. DNA was purified from cervical brush samples by a simple modification (squeezing the cytobrushes on the side of the tube, and freezing times were increased to 24 hours from 2 hours) of the freeze-thaw method of described by Jacobs et al in Jacobs et al J Clin Microbiol 35 791-5 (1997). Epithelial cells obtained from the cytobrush samples were pelleted by centrifugation and re-suspended in 1 ml 10 mM Tris pH 7.4, and frozen at −70° C. for 24 hours. A 100 μl aliquot was thawed, boiled for 10 minutes; chilled on ice; spun at 13,000 rpm in a microfuge for 3 minutes; and supernatant decanted and stored. DNA was extracted from biopsy material using a modification of the above technique, in which samples were incubated in 1 ml 10 mM Tris HCl pH7.4 containing 10 mg/ml proteinase K (from Sigma, UK) for 1 hour at 56° C. before boiling.

PCR EIA—Comparative Method

PCR-EIA was performed as described by Jacobs et al (q.v.).

VESPA Method

Primer Design

Table 2 shows the sequences of the ten self-probing amplicon primers used in this study. All primers were synthesised by Oswel Research Products, Southampton SO16 7PX, UK.

All self-probing amplicon primers were purified by double HPLC. Standard primers were purified by gel filtration. Primers were designed to detect the HPV L1 gene. Primer locations varied between types but were positioned approximately between 6600 and 6750 bp.

The forward primer sequence of each self-probing amplicon is type-specific, and is located at the same sequence position as that of the GP6⁺ primer described by Jacobs et al (q.v.). Self-probing amplicon probe sequences were designed by aligning the L1 open reading frames (ORF) of twenty common HPV types (HPV-6, 11, 16, 18, 31, 33, 39, 40, 42, 43, 44, 45, 51, 52, 56, 58, 59, 66 and 68) (http://hpv-web.1an1.gov). The areas of greatest sequence variation adjacent to the GP6⁺ primer binding site of Jacobs et al (q.v.) were selected as the probe target binding site. The probe sequence of these primers was checked against 70 common papillomavirus sequences and no significant homology was found. The SC16 primer component comprises the known GP6+ sequence. The reverse primer target sequence is the GP5+ sequence of Jacobs et al (q.v.).

TABLE 2

| Sequences of VESPA Primers. | | |
|---|---|---|
| Typing | SEQ ID | |
| Primers | NO: | Sequence |
| HPV-6 Sc6 | 1 | CCGCGGATAAAGAGTACATGCGTCCGCGG-MR-HEG-GAAAAATAAATTGTAAATCATACTC |
| HPV-11 Sc11 | 2 | CCCGCGGCAGATTATAAGGAATACATGCCCGCGGG-MR-HEG- |
| | | GAAAAATAAACTGTAAATCAAACTC |
| HPV-16 Sc16 | 3 | CCGCGGAGTACCTACGACATGGGCCGCGG-MR-HEG-GAAAAATAAACTGTAAATCATATTC |
| HPV-18 Sc18 | 4 | CCGCGGCAGCAGTATAGCAGACATGGCCGCGG-MR-HEG- |
| | | GAAAAATAAACTGCAAATCATATTC |
| HPV-31 Sc31 | 5 | CCCGCGGGAGTATTTAAGACATGGTGCCGCGGG-MR-HEG- |
| | | GAAATATAAATTGTAAATCAAATTC |
| HPV-33 Sc33 | 6 | CCGCGGCTTTATGCACACAAGAACCCGCGG-MR-HEG-GAAAAACAAACTGTAGATCATATTC |
| HPV-39 Sc39 | 7 | CCCGCGGAATATACCAGGCACGTGCCGCGGG-MR-HEG-GAAATATAAATTGTAAATCATACTC |
| HPV-51 Sc51 | 8 | CCCGCGGGCAATATATTAGGCATGGGCCGCGGG-MR-HEG-AAAAATAAATTGCAATTCATACTC |
| HPV-56 Sc56 | 9 | CCGCGGTCAGTACCTTAGACATGTGCCGCGG-MR-HEG-GAAAAACAAATTGTAACCCATATTC |
| Reverse | | 5′-TTTGTTACTGTGGTAGATAC |
| Primer-GP5+ | | |

HPVn = human papillomavirus n; Sc = self-probing amplicon; MR = Fluorescence quencher methyl red; HEG = PCR blocker = hexethyl glycol. Sequences in bold typeface are novel probe sequences.

Primer Validation—VESPA v Reference Cell Lines

The HPV 16 and 18 primers were tested for specificity using reference cell lines with integrated HPV DNA. The Caski cell line contains 60-600 copies of the HPV-16 ORF per cell, and the HeLa cell line contains 10-50 copies of the HPV-18 L1 ORF.

FIG. 1a shows the results of PCR reactions using the Sc16 self-probing amplicon primer (designed for detection of HPV-16 DNA), and DNA extracted from the HPV-16 positive Caski cell line, DNA from the HPV-18 positive HeLa cell line and a negative control (no DNA). A significant increase in fluorescence was only detected with the HPV-16 containing Caski DNA.

FIG. 1b shows a similar experiment using the Sc18 self-probing amplicon primer in place of Sc16. Here, significant fluorescence was only detected with the HPV-18 positive HeLa cell line. The primers were then used to detect HPV-16 and -18 in clinical samples previously typed using PCR-EIA (FIG. 2c and FIG. 2d—see below).

Detection of HPV by VESPA

PCR amplification of 1 μl aliquots of DNA solution were performed using 0.5 μM self-probing amplicon primer and 0.5 μM GP5⁺ reverse primer as described by Jacobs et al (q.v.) in a total reaction volume of 10 μl. Reactions were performed using a Light Cycler (available from Bio/Gene, Kimbolton, Cambs, PE18 0NJ or Roche Diagnostics Ltd, Bell Lane, Lewes, East Sussex BN7 1LG) and run for 100 cycles under the following cycling parameters: 96° C.-1s, 40° C.-5s, 72° C.-1s. Reaction conditions were as follows: 200 μM dNTP's, 4 mM MgCl₂, 50 mM Tris HCl pH 8.9, 10 mM ammonium sulphate, 0.1% Tween™ 20, bovine serum albumin 250 ng/μl, and 0.5 U/μl Taq polymerase (from Advanced Biotechnologies, Epsom, Surrey, UK). Fluorescence was detected in channel one (530 nm) at 40° C.

PCR control reactions were carried out as above but included 1 μl SYBR Gold (from Bio/Gene, Kimbolton, Cambs, PE18 0NJ). Self-probing amplicon control reaction (negative controls) contained 1 μl H₂O in place of DNA.

The VESPA technique was then extended to enable detection of HPV types-6, -11, -31, -33, -39, -51 and -56. As there are no commercially-available cell lines containing these HPV types, primer specificity was validated using clinical samples previously tested using PCR-EIA as shown below in Table 5, with reference to Example 3. FIG. 2(a-h) shows positive results from typing reactions for HPV-6, -11, -16, -18,-31, -33, -39, and -51, respectively.

### EXAMPLE 2

### Viral Load Determination Using VESPA

A theoretical advantage of self-probing amplicon PCR is its ability to determine viral load. Shown, in FIG. 3, are the results of Sc16 typing reactions performed using a dilution series of the SiHa cell line (one to two copies of HPV-16 per cell). The dilution series from 50,000 to 500 HPV copies per cell were clearly distinguishable, and the signal for HPV-16 remained positive in the sample containing a single copy of HPV-16 DNA.

In order to explore the possibility of establishing a technique capable of determining viral load per cell, a primer was designed to detect (human) beta-globin DNA (ScBG, SEQ ID NO: 11 in Table 3).

TABLE 3
HPV-16 Viral Load - Sequences of VESPA Primers.

| Beta- | | |
|---|---|---|
| Globin | | |
| Detection | SEQ ID | |
| Primer | NO: | Sequence |
| ScBG | 11 | FAM-CCGCGGATGGTGTCTGTTTGAGCCGCGG- |
| | | MR-HEG-ACACAACTGTGTTCACTAGC |
| Reverse | | 5′-GAAACCCAAGAGTCTTCTCT-3′ |
| Primer | | |

Sc = self-probing amplicon; FAM = Fluorophore = 6-carboxyfluorescein. MR = Fluorescence quencher = Methyl Red; HEG = PCR blocker = hexethyl glycol.
Sequences in bold typeface are novel probe sequences

FIG. 4 shows the results of an experiment conducted using a self-probing amplicon designed to detect the human beta-globin gene over the same dilution series of SiHa cells used above for HPV-16. Once again, the signal remained positive down to a single cell and is quantitative at and above 50 copies per cell. FIG. 5 shows a plot of the ratio of the fluorescence produced by Sc16 and ScBG against the logarithm of the viral copy number per cell of target DNA. A range of copy numbers per cell may be calculated from a cell line containing a fixed copy number, by calculating the ratio of two different dilutions. For example, the Sc16 Fₘₐₓ value for 5000 cells can be divided by that for the ScBG Fₘₐₓ for 100 cells to obtain a value for a notional cell line containing 50 copies per cell (5000/100).

Disease grade was determined by cytology. VL ratio (Viral Load) was measured in RSU Relative Self-probing amplicon Units) by dividing Sc16 Fₘₐₓ by ScBG Fₘₐₓ. Viral copies per cell were estimated using the standard curve shown in FIG. 5

TABLE 4
Viral Load Data

| | | HPV- | Disease | VL ratio | Av. Viral Copies |
|---|---|---|---|---|---|
| | Sample | Type | Grade | (RSU) | per cell |
| 1 | 2885C | 16 | Normal | −0.3508 | Undetectable |
| 2 | 2503F | 16 | Normal | −0.1204 | Undetectable |
| 3 | 0155H | 16 | Normal | −0.1007 | Undetectable |
| 4 | LD20 | 6,16,39 | CIN2 | −0.0242 | Undetectable |
| 5 | 0119D | 16 | Normal | −0.0166 | Undetectable |
| 6 | SJ | 16 | Ca vagina | 0.0095 | 0.042 |
| 7 | LD49 | 16 | CIN2-3 | 0.0331 | 0.047 |
| 8 | AD | 16 | CIN2-3 | 0.0550 | 0.052 |
| 9 | LD19 | 16 | CIN2 | 0.0697 | 0.056 |
| 10 | LD15 | 16 | CIN3 | 0.0750 | 0.057 |
| 11 | LD25 | 16,31 | CIN3 | 0.0786 | 0.058 |
| 12 | LD24 | 16 | CaCx | 0.1641 | 0.088 |
| 13 | LD50 mm | 16 | CIN3 | 0.4945 | 0.441 |
| 14 | 2503F | 16 | CIN2-3 | 0.5965 | 0.724 |
| 15 | LD45 | 16 | CIN2-3 | 0.7601 | 1.603 |
| 16 | 0093A | 16 | CIN2-3 | 0.8749 | 2.805 |

Shown in Table 4 are the results of applying this viral load determination technique to 16 clinical samples previously found (by Jacobs et al, q.v.) to be HPV-16-positive using PCR-EIA. The most striking finding from these experiments is that the four cervical smears with normal cytology have low viral loads using VESPA, and the only other sample with a low viral load, but significant neoplasia, is co-infected with HPV-6 and HPV-39. The preliminary viral load data shown in Table 4 suggest that the presence of cervical neoplasia might correlate with viral load.

### EXAMPLE 3

### Comparison of VESPA With PCR-EIA

In order to test the suitability of VESPA for HPV typing from cervical smears in the clinical setting, the self-probing amplicon primers were used to test 108 samples previously HPV typed (Jacobs et al q.v.), using PCR-EIA. In order directly to compare the two techniques, DNA extraction was performed using the freeze-thaw method described by Jacobs et al, q.v. This technique is sub-optimal for PCR amplification using self-probing amplicon primers. The less efficient freeze-thaw DNA extraction method was used in this study, since this method is recommended for PCR-EIA and we wished to compare PCR-EIA and VESPA using identical DNA samples. The results of these experiments are shown in Table 5.

Selected positive sample types have subsequently been confirmed by direct sequencing (data not shown). The overall concordance between VESPA and PCR-EIA is 94%, with a kappa value of 0.89 indicating good agreement. Indeed, a similar concordance figure was achieved when PCR-EIA was performed on identical samples by different reference laboratories described by Jacobs et al q.v. Of 108 samples, VESPA failed to detect five incidences of HPV-16 and one of HPV-18. There were no false positives.

There are several explanations for the small number of remaining discrepancies observed between VESPA and PCR-EIA. First, the results produced by PCR-EIA might be false positives. There is evidence from the study comparing PCR-EIA in several different laboratories that it is prone to the occasional false positive. The fact that four of the six discrepant samples have normal cytology supports this supposition. Of the remaining two, one has low-grade lesions and for the other we have no clinical data. Secondly, the samples may be positive for HPV but contain an intra-typic HPV variant containing polymorphism within the probe-binding site. Self-probing amplicon probes discriminate between sequences on the basis of a single base change, whereas PCR-EIA probes are more tolerant of sequence variation. In this regard, it may be relevant that 5/6 discordant samples were obtained from West Africa, where there is likely to be more variation within the probe binding site.

TABLE 5
Comparison of PCR-EIA and VESPA

| | | | | KAPPA |
|---|---|---|---|---|
| Sample Type | HPV Type(s) | PCR-EIA | VESPA | VALUES |
| Cell Line | 16 | 2 | 2 | n/a |
| (n = 3) | 18 | 1 | 1 | |
| Biopsy | 6 | 1 | 1 | n/a |
| (n = 3) | 11 | 2 | 2 | |
| UK Cervical Samples | 16 | 8 | 7 | |
| | 33 | 2 | 2 | |
| (n = 54) | 16,31 | 1 | 1 | |
| | 16,39 | 1 | 1 | 0.95 |
| | 6,16,39 | 1 | 1 | |
| | 33,51 | 1 | 1 | |
| | neg | 40 | 41 | |
| African Cervical | 11 | 4 | 4 | |
| Samples | | | | |
| (n = 48) | 16 | 7 | 3 | |
| | 16,31 | 1 | 1 | |
| | 18 | 6 | 5 | |
| | 31 | 2 | 2 | 0.79 |
| | 33 | 2 | 2 | |
| | 39 | 2 | 2 | |
| | 51 | 3 | 3 | |
| | 56 | 3 | 3 | |
| | neg | 18 | 23 | |
| | | | Overall | 0.89 |
| | | | (n = 108) | |

k-values were calculated as described by Armitage et al in Statistical Methods in Medical Research, Third Edition (Oxford; Blackwell Scientific Publications)

### EXAMPLE 4

### VESPA to Detect Integration Status—Calculation of E6/E7:E1/E2

Self-probing amplicon sequences designed for the assessment of HPV integration state are shown in Table 6. They are used in accordance with the method described in the Examples above. Integration status is measured as described hereinabove.

TABLE 6
Integration Primers

| | SEQ | |
|---|---|---|
| | ID | |
| Primer | NO: | Sequence |
| Sc16- | 12 | 5′-FAMCCGCGGGCAAAGAGTAATCATTACCGCGG-MR |
| E1mid | | HEG-CAGAATGGATACAAAGACAAACAGT-3′ |
| RP16 E1 | | 5′-GCGCATGTGTTTCCAATAGTCTA-3′ |
| Sc16-E2 | 13 | 5′-FAMCCCGCGGTTGTCATATAGACATATCATTTT- |
| | | CATCCGCGGG-MR-HEG- |
| | | CAACGTTTAAATGTGTGTCAGGA-3′ |
| RP16 E2 | | 5′-AGACACACAAAAGCACACAAAAGC-3′ |
| Sc16 E6 | 14 | 5′-ROXCCGCFAMTGGCGAATGTCTACATATCATGGC- |
| | | CCAGCGG-MR-HEG- |
| | | AAGTTACCACAGTTATGCACAGAGC-3′ |
| RP16 E7 | | 5′-GAGAACAGATGGGGCACACAAT-3′ |
| Sc18 | 15 | 5′-FAMCCCGCGGTCGGTGTCTCCATGTTGCCGCGGG- |
| E1mid | | MR-HEG-AGTAATGGGAGACACACCTGAGT |
| RP18 E1 | | 5′-AGTGGTCTATGATTTTGTCCTGCA-3′ |
| Sc18-E2 | 16 | 5′-FAMCCGCGGTACATTGTCATGGTCTATGAT- |
| | | CCGCGG-MR-REG-GCAGACACCGAAGGAAACCC-3′ |
| RP18-E2 | | 5′-GCATACACAAAAGCAAAATAAAAAA-3′ |
| Sc18-E6 | 17 | 5′ROXCCGCFAMTGGCTGGAATGCTATATCATGCCA- |
| | | GCGG-MR-HEG-ACCCAGAAAGTTACCACAGTTAT-3′ |
| RP18-E7 | | 5′CCGTCTGTACCTTCTGGATC-3′ |

Key:
Sc = self-probing amplicon; RP = reverse primer
ROX = ROX; FAM = Fluorophore = 6-carboxyfluorescein
MR = Fluorescence quencher = Methyl Red
HEG PCR blocker = hexethyl glycol
Bold typeface indicates probe and primer sequences, respectively.

### EXAMPLE 5

### VESPA Using Tailed Primers for Degenerate HPV Detection

A suitable strategy comprises the following steps, illustrated by FIG. 6:

1. A primer containing a ‘designer tail’ is used to amplify viral DNA (if present). A standard reverse primer is also included.

2. Amplification proceeds as in a standard reaction.

3. After the first round of amplification, the ‘designer’ sequence has no complementary sequence.

4. After the second round of amplification, the ‘designer’ sequence is incorporated and is amplified in each round.

5. In a separate reaction, this amplification can then be detected using a self-probing amplicon comprising a primer targeting the ‘designer’ sequence and a probe for detecting the consensus primer. It may be necessary to use two self-probing amplicons with a slight overhang into the viral DNA to avoid unacceptable background fluorescence caused by primer dimer.

Using a method analogous to that described in previous Examples, the following tailed primers were used to detect degenerate HPV (Table 7):

TABLE 7
Tailed Primers

| | SEQ ID | |
|---|---|---|
| Primer | NO: | Sequence |
| Tailed | 18 | ATGTGGAAACATGCATGGCGAGATGAAAAATAAACTG |
| GP6+ | | TAAATCATATCT |
| ScDGi | 19 | FAM-CCGCGGGAAGAATATGATTTACACCGCGG-MR- |
| | | HEG-GTGGAAACATGCATGGCGAC |
| ScDGii | 20 | FAM-CCGCGGGAGGAATATGATTTACACCGCGG-MR- |
| | | HEG-GTGGAAACATGCATGGCGAC |

Key:
Sc = self-probing amplicon; FAM = Fluorophore 6-carboxyfluorescein
MR = Fluorescence quencher = Methyl Red; BEG = PCR blocker = hexethyl glycol
Sequence in bold typeface is novel tail region. Both primer and probe regions are also novel.

FIG. 7 demonstrates the ability of the degenerate self-probing amplicon mix to detect HPV types-6, -16 and -18. Comparisons may be performed using samples pre-amplified under conditions described by Jacobs et al (qv). Interestingly, two of these samples were barely visible after agarose gel electrophoresis with ethidium bromide staining, suggesting that the VESPA approach produces the expected improvement in sensitivity.

### EXAMPLE 6

### VESPA Applied to SV40

DNA Extraction

Ten sequential 5 μM sections of paraffin-embedded mesothelioma biopsies were cut, taking precautions to avoid block-to-block contamination. The sections were placed in microfuge tubes and treated with xylene for 10 mins in order to remove paraffin, then washed twice with ethanol and dried. 400 μl of digestion buffer containing 200 μg/ml of proteinase K were added and the specimens digested overnight at 55° C. Proteinase K was then inactivated by heating to 95° C for 10 mins. The digests were further processed for DNA extraction using phenol/chloroform/isoamyl alcohol. DNA was precipitated with ethanol, centrifuged and air dried. Further de-salting and concentration of DNA was performed using Centricon 30 filters (Millipore, Bedford, Mass., USA). The pure DNA was quantified spectrophotometrically. All samples tested positive for beta-globin amplification (Muggleton-Harris et al. Hum Reprod 10 183-92 (1995)), indicating PCR viability.

Primer Design

Primers were designed by aligning the sequence of each virus. (http://www.ncbi.nlm.nih.gov/PubMed/). SV40 Accession Number J02400; JC Accession Number J02226/7; BK Accession Number NC 001538. For the first round amplification, several primers were designed, throughout the large T antigen gene (see Table 1), using established, historical primer sites. For the first round amplification step, because of the substantial degradation of the DNA found in most samples, it was preferred to use primers capable of targeting short DNA fragments in which the target sites were close together. Self-probing amplicon primer and probe binding sites were designed to target areas of least homology between these primer sites.

### Table 8: Sequences of SV40-VESPA Primers

All self-probing amplicon primers were purified by double hplc. Standard primers were purified by gel filtration. Primers were designed to detect the large T antigen.

Key:

FAM=Fluorophore=6-carboxyfluorescein

MR=Fluorescence quencher=Methyl Red

HEG=PCR blocker=hexethyl glycol

The approximate position of primers and probes is also shown. P1 indicates a forward primer; P2 and P3 are reverse primers.

TABLE 8

| | | |
|---|---|---|
| Name | Location | Sequences of SV40-VESPA Primers & Probes |

| | | |
|---|---|---|
| ScsSV40 | 4361- | a g ca t g a c t c a a a a a a c t t a g c a a t t c t |
| primer | 4388 | |
| ScJC primer | 4361- | t t ct c a t t a a a t g t a t t c c a c c a g g a t t |
| | 4388 | |
| ScBK primer | 4347- | a g ct t g a c t a a g a a a c t g g t g t a g a t c a |
| | 4374 | |
| ScSV40 | 4390- | a c ccc a a g g a c t t t c c t |
| probe | 4406 | |
| Sc JC probe | 4390- | c c ta t g g a a c a g a t g a a t g |
| | 4406 | |
| ScBK probe | 4376- | a cc c t a a a g a c t t t c c c |
| | 4392 | |
| SV40 P1 | 4318- | t t tt c c t t t g t g g t g t a a a t |
| | 4337 | |
| SV40 P2 | 4921- | a c tt t g g a g g c t t c t g g g a t g c a a c |
| | 4945 | |
| SV40 P3- | 4371- | a g at t c c a a c c t a t g g a a c t g a t |
| | 4398 | |
| SV40 P4 | 4453- | g a gg c t a c t g c t g a c t c t c a a c a t |
| | 4476 | |
| JC P1 | 4318- | t t tc a t c a t c a c t g g c a a a c |
| | 4337 | |
| JC P2 | 4921- | c a tgg g g g a a c a t t c c t g t c a t g a g |
| | 4945 | |
| JC P3 | 4371- | g t gt g a c t t a a c c c a a g a a g c t c |
| | 4398 | |
| JC P4 | 4453- | t t t gc a g g g c a t t t t g t t t t t t a c |
| | 4476 | |
| BK P1 | 4550- | t t tt c t t t a g t a g t a t a c a c |
| | 4574 | |
| BK P2 | 4453- | t g ga a c c t g g a g t a g c t c a g a g g t t |
| | 4476 | |
| BK P3 | 4908- | c c ta t g g a a c a g a a g a g t g g g a g |
| | 4932 | |
| BK P4 | 4439- | g a a gc a a c a g c a g a t t c t c a a c a c |
| | 4463 | |

Assay Conditions

All amplifications were carried out using good laboratory practise, filter tips and in a laboratory not previously exposed to polyomaviruses.

First Round Amplification (Pre-amplification).

Each reaction was set up three times with appropriate primer pairs in order to test for SV40, JC and BK.

5 μl of each sample per reaction was pre-amplified under the following conditions; 0.5 μM P1 and P3 (see Table 8), 2.5 mM MgCl₂, 200 μM dNTPs, 1.25 U of Taq polymerase (Advanced Biotechnologies, Epsom, Surrey, UK) and 5 μl of 10× reaction buffer (Perkin-Elmer Gold) in a total reaction volume of 50 lμ.

Each sample was also SV40-typed under the conditions described above and using the P1-P2 primer pair (Table 8).

Second Round Amplification (Amplicon Detection).

Assay set-up was performed in a laboratory physically separated (100 m) from that used for the first round amplification.

Amplicon detection was performed in two different real-time PCR machines:

(i) LightCycler (Roche Diagnostics Ltd, Bell Lane, Lewes, East Sussex BN7 1LG) and

(ii) iCycler (Bio-Rad Laboratories Ltd., Bio-Rad House, Maylands Avenue, Hemel Hempstead, Herts. HP2 7TD); and under slightly different conditions.

PCR amplification of 1 μl aliquots of DNA (2 μl iCycler) solution were performed using 0.5 μM Scorpion primer (see Table 1) and 0.5 μM reverse primer in a total reaction volume of 10 μl (20 μl iCycler). Reaction conditions were as follows: 200 μM dNTPs, 4 mM MgCl₂, 50 mM Tris HCl pH 8.9, 10 mM ammonium sulphate, 0.1% Tween 20, bovine serum albumin 250 ng/μl, and 0.5 U/μl Taq polymerase (Advanced Biotechnologies, Epsom, Surrey, UK). Fluorescence was detected in channel one/530 nm (FilterSet4 iCycler) and at 40° C. (45° C. iCycler). Self-probing amplicon control reaction (negative controls) contained H₂O in place of DNA. All primers were synthesised by Oswel Research Products, Southampton SO16 7PX, UK.

Cycling Parameters

Pre-Amplification

94° C. for 180 s followed by 30 cycles of 94° C.-60 s, 52° C.-60 s and 72° C.-60 s and finally 72° C.-300 s.

Amplicon Detection

(i) LightCycler. 100 cycles of 96° C.-1 s, 40° C.-5 s, 72° C.-1 s.

(ii) iCycler. 95° C. for 180 s followed by 40 cycles of 95° C.-30 s, 45° C.-30 s, 72° C.-15 s.

Results

Shown in FIGS. 10a-10c are the results of challenging the self-probing amplicon designed for the detection of SV40 (ScSV40), that designed for the detection of JC virus (ScJC) and that designed for the detection of BK virus (ScBK) with pre-amplified cell line DNA, mesothelioma-derived DNA and water in a LightCycler real time fluorimeter. A significant increase in fluorescence is observed only when a specific self-probing amplicon encounters a cognate virus. Background levels of fluorescence due to non-cognate virus remains low in all cases bar ScBK when challenged with JC virus.

FIGS. 10d-10f show equivalent reaction profiles when analysed using an iCycler real time fluorimeter. FIGS. 10e and 10f do not show reaction profiles produced by samples. Fluorescence is strong when a specific self-probing amplicon is presented with cognate virus and weak with non-cognate virus. Background fluorescence due to non-cognate virus is, however, increased in this series of experiments.

Mesothelioma viral typing results, using all three self-probing amplicon, two ScSV40 target amplicons and two different modes of amplicon analysis, are shown in Table 9.

### Table 9: Analysis of Mesothelioma-Derived DNA Using SV40-VESPA

Positives were assigned to those reactions producing at least three times background fluorescence (ie 3× that produced by a blank reaction containing only water or background fluorescence due to non-cognate virus, whichever is higher).

TABLE 9
Analysis of Mesothelioma-derived DNA using SV40-VESPA
Positives were assigned to those reactions producing at least three times background
fluorescence (ie 3 x that produced by a blank reaction containing only water or
background fluorescence due to non-cognate virus, whichever is higher).

| Scorpion Type | SV4D | | | | JC | BK |
|---|---|---|---|---|---|---|
| Amplicon analysed | | P1-P2 | P1-P3 | | P1-P3 | P1-P3 |
| Mode of Analysis | Rodu et al | LightCycler | LightCycler | iCycler | LightCycler | LightCycler |
| Sample Name | | | | | | |
| Positive Control | | pos | pos | pos | pos | pos |
| Blnk | | neg | neg | neg | neg | neg |
| 1 | | neg | pos | pos | neg | neg |
| 2 | | neg | neg | neg | neg | neg |
| 3 | | neg | insuf | insuf | insuf | insuf |
| 4 | | neg | insuf | insuf | insuf | insuf |
| 5 | | neg | pos | pos | neg | neg |
| 6 | | pos | pos | pos | neg | neg |
| 7 | | neg | pos | pos | neg | pos |
| 8 | | neg | pos | pos | neg | neg |
| 9 | | neg | pos | pos | neg | neg |
| 10 | | neg | neg | neg | neg | neg |
| 11 | | neg | pos | pos | neg | neg |
| 12 | | neg | neg | neg | neg | neg |
| 13 | | neg | pos | pos | pos | neg |
| 14 | | neg | neg | neg | neg | neg |
| 15 | | neg | pos | pos | neg | neg |
| 16 | | neg | neg | neg | neg | neg |
| 2059 | | neg | neg | neg | neg | neg |
| W335 | | neg | pos | pos | neg | neg |
| GFPM | | neg | pos | pos | pos | neg |
| S84 | | neg | pos | pos | pos | neg |
| 10684 | | neg | pos | pos | neg | neg |
| 495-2905 | | neg | pos | pos | neg | neg |
| 6813 | | neg | pos | pos | neg | neg |
| 36/96-2 | | neg | neg | neg | neg | neg |
| 2341 | | neg | neg | neg | neg | neg |
| 594 | | neg | neg | neg | neg | neg |
| 286 700 6 | | neg | neg | neg | neg | neg |
| 286 700 4 | | neg | pos | pos | neg | neg |
| F96 3-5 | | neg | neg | neg | pos | neg |
| 951-4-6 | | neg | pos | pos | neg | neg |
| P9800162F | | neg | pos | pos | neg | neg |
| 184/92H | | neg | pos | pos | neg | neg |
| P96/3473 | | neg | pos | pos | neg | neg |
| 634/94A | | neg | pos | pos | pos | neg |
| 103/95 | | neg | pos | pos | pos | neg |
| BP 9459 | | neg | pos | pos | neg | neg |
| Positive Control | | pos | pos | pos | pos | pos |
| Blnk | | neg | neg | neg | neg | neg |
| Positive Control | | pos | pos | pos | pos | pos |
| Blnk | | neg | neg | neg | pos | neg |
| PM 149/9322 | | neg | pos | pos | neg | neg |
| 6656/98B | | neg | pos | pos | neg | neg |
| 96N 129B | | neg | pos | pos | pos | neg |
| 10 330/94 R2 | | neg | pos | pos | neg | neg |
| 95-67-P | | neg | neg | neg | neg | neg |
| D94-7M | | neg | pos | pos | neg | neg |
| S84/93 | | neg | pos | pos | pos | neg |
| 634/94A | | neg | neg | neg | neg | neg |
| 95/10684I | | neg | neg | neg | neg | neg |
| 495-2905 | | neg | neg | neg | neg | neg |
| P94-212 | | neg | pos | pos | neg | neg |
| M93-268-1 | | neg | neg | neg | neg | neg |
| F98-29-4 | | neg | pos | pos | neg | neg |
| 5326-91 | | neg | neg | neg | neg | neg |
| F237-91 | | neg | neg | pos | neg | neg |
| 119M-94 | | neg | neg | pos | neg | neg |
| 3226-91 | | neg | neg | pos | neg | neg |
| A-352-91 | | neg | neg | pos | neg | neg |
| 630-91-5 | | neg | neg | neg | neg | neg |
| 3009-91 | | neg | neg | pos | neg | neg |
| A17886/95 | | neg | neg | pos | neg | neg |
| 95N26 | | neg | neg | pos | neg | neg |
| 9839/97 | | neg | pos | pos | neg | neg |
| 537-94-7 | | neg | pos | pos | pos | neg |
| 96/746A | | neg | pos | pos | pos | neg |
| 14839/94 8 | | neg | pos | pos | neg | neg |
| 128/93 | | neg | pos | pos | pos | neg |
| A3087-91 | | neg | pos | pos | neg | neg |
| 7327/94 | | neg | pos | pos | pos | neg |
| 93/3012 | | neg | pos | pos | pos | neg |
| TP 247/94 | | neg | neg | neg | pos | neg |
| 97-0818D | | neg | pos | pos | neg | neg |
| 10947/94 | | neg | pos | pos | neg | neg |
| 93-300 | | neg | pos | pos | neg | neg |
| M93606A | | neg | neg | neg | neg | neg |
| P93 6249/4 | | neg | pos | pos | neg | neg |
| B476A93 | | neg | neg | pos | neg | neg |
| P2 54/92 | | neg | neg | neg | neg | neg |
| E9254/92/10 | | neg | neg | neg | neg | neg |
| A414/1/92 | | neg | pos | pos | neg | neg |
| Positive Control | | pos | pos | pos | pos | pos |
| Blnk | | neg | neg | neg | neg | neg |
| Positive | | 1 | 44 | 52 | 14 | 1 |
| Negative | | 75 | 30 | 22 | 60 | 73 |
| % incidence | | 1.30% | 59% | 70% | 19% | 1.40% |

These are the typing results of 76 archival mesothelioma DNA samples derived from paraffin embedded blocks. There are several observations of note. First, analysis targeting a 650 bp amplicon between P1 and P2 (see Table 8) produces a single positive, whereas analysis targeting a 230 bp amplicon between P1 and P3 produces 44 positives. This observation is most likely due to the fact that often only short sections of DNA may be recovered from paraffin-embedded blocks but may also reflect the fact that SV40 has become integrated into the host genome in these samples with concomitant DNA deletion.

Secondly, comparing SV40-VESPA and established methodology reveals an increase in the number of positives observed suggesting an increase in sensitivity. This comparison is dependent on the mode of fluorescence measurement. SV40 incidences of 1.3% were found when analysing amplicon P1-P2 (650 bp); 59% when analysing amplicon P1-P3 (230 bp) using a LightCycler; and 70% when analysing amplicon P1-P3 using an iCycler. Thus, data produced using an iCycler reports nearly 20% more positives than that reported by a LightCycler. Background fluorescence is, however, increased using the iCycler. This increase in the observed sensitivity of the iCycler may be due to longer reaction times (2 hours v 1 hour) or larger sample volume (2 μl v 1 μl). The iCycler also displays advantages in terms of throughput (96 v 32). There were no samples reported positive using the iCycler and negative using the LightCycler. These results indicate that the LightCycler is better suited to specific analyses, whereas the iCycler is better suited to sensitive analyses.

Thirdly, there was one false positive observed with ScJC, which occurred in a control negative blank reaction. This observation highlights the sensitivity of the assay and the need for scrupulous assay technique and caution when interpreting viral analyses of this sort. This problem has been encountered before in laboratories responsible for testing UK polio vaccines with no convincing explanation as to the source of contamination. The fact that this false positive occurred in an experiment that contained five other blanks, all producing the expected negative result, suggests this was an isolated case. Also, the fact that the laboratory used has not been previously exposed to polyomaviruses suggests that this blank is positive as a consequence of being located adjacent to two positive controls. Thus, sample carryover may be responsible.

The incidence of SV40 found has a maximum value of 70% and thus lies within, but towards the upper limit of, previously-reported ranges. JC and BK virus are estimated to occur in 60% of the paediatric population with over 70% of adults having JC and BK antibodies. An incidence of 33% has been reported for JC virus in tumours of the central nervous system. The incidence of JC and BK found was 19% and 1.4%, respectively.

Estimation of viral load in these samples is complicated by the use of nested PCR in which reactions may reach ‘limiting conditions’ during the first round of amplification. SV40-VESPA is capable of typing cell line DNA in a one round amplification (data not shown). Thus, those samples that are strongly positive in the nested screen could be re-analysed for viral load in a one round PCR using internal standards for human genomic DNA. The data show DNA samples with ScSV40 fluorescence in the range 1.4 to 26.5, consistent with there being a large range of viral loads present in the mesothelium biopsies from which they were derived.

### CONCLUSION

FIGS. 10a-10f demonstrate that the present invention results in an assay capable of producing strong signals in response to cognate virus and of discriminating cognate virus from large concentrations of highly homologous non-cognate virus. The one possible exception to this statement is the observation of significant background fluorescence when ScBK is challenged with pre-amplified JC virus. Although still acceptable at around 10% of the positive control value, there is the possibility of assigning JC virus as a false positive BK virus. This observation does not affect the current study, since all samples were typed for JC virus and the one sample positive for BK was JC negative. However, all BK positives produced using this technique must be interpreted with caution and ideally typed for JC virus. It is worth re-emphasising, however, that ScBK background fluorescence is only observed in the presence of pre-amplified JC DNA at high concentration.

Therefore, the central findings of this experiment are:

(i) The incidence of SV40 is dependent on size of amplicon probed;

(ii) The incidence of SV40 is dependent on the mode of amplicon analysis; and

(iii) The rate of infection of SV40 in archival mesothelioma tissue is in the range of from 58% to 70%.

### EXAMPLE 7

### VESPA in HPV Associated with RRP

Clinical Samples

Patients were recruited by contacting all Ear, Nose and Throat surgeons throughout Wales and all those in England known to be actively engaged in recurrent respiratory papillomatosis (RRP) research. All cases of RRP, as confirmed by characteristic histological changes on biopsy and/or those requiring more than 2 surgical interventions, were identified. Informed consent was obtained from all subjects. Biopsies were collected, with informed consent, from patients undergoing treatment for Recurrent Respiratory Papillomatosis at the University Hospitals of Wales, Cardiff and Sheffield.

Disease Severity

The grade of clinical disease was assigned using the parameters outlined by Derkay et al in Laryngoscope 108(6) 935-7 (1998); this equation accounts for both the number and time interval between surgeries.

DNA Purification

DNA was extracted from biopsies by incubating approx 1 mm³ of biopsy material in 1 ml 10 mM Tris hydrochloride pH7.4 containing 0.5 mg/ml proteinase K (Sigma, UK) for 1 hour at 56° C. 100 μl aliquots were then taken and boiled for 10 minutes, cooled on ice for 5 minutes, spun at 13,000 rpm for 3 minutes (in order to remove particulate matter) and decanted into fresh tubes.

PCR EIA

In order to confirm HPV types, the reference technique of Jacobs et al (qv) for PCR-EIA was performed on all samples as described in Example 3.

Results

The viral loads presented in Table 10 were calculated as described in Example 2. Briefly, the ratio of the fluorescence signals produced using a cognate HPV-self-probing amplicon and that of a self-probing amplicon designed to detect a human genomic beta-globin housekeeping gene was calculated.

Although there are clearly examples of patients with high viral load and mild disease (NL) and vice versa (DN), a patient who is in remission had the lowest viral load and the most severe patient had the highest viral load. This study suggests that the most significant prognostic marker in RRP is HPV-type; HPV-6 leads to mild and HPV-11 to aggressive disease.

TABLE 10

| Sample | | | | Viral Load |
|---|---|---|---|---|
| Name | HPV Type | Onset | Disease Grade | Ratio |
| MW | neg (was 6) | JORRP | Regression | −0.02 |
| TW | 6 | JORRP | Aggressive/Mild | 1.15 |
| JS | 6 | JORRP | Aggressive | 1.34 |
| DF | 6 | AORRP | Mild | 3.74 |
| MP | 6 | AORRP | Mild | 4.25 |
| RM | 6 | JORRP | Aggressive | 4.63 |
| DN | 11 | JORRP | Aggressive | 0.92 |
| JJ | 11 | JORRP | Aggressive | 2.41 |
| NL | 11 | JORRP | Mild | 6.58 |
| DH | 11 | JORRP | Aggressive | 24.12 |

1-55. (canceled)

56. A self-probing amplicon comprising a nucleic acid sequence selected from the group consisting of:
(i) SEQ IDs NOS: 1 to 9;
(ii) SEQ ID NO: 11;
(iii) SEQ ID NOS: 12 to 17;
(iv) SEQ ID NOS: 18 to 20; or
(v) the group of self-probing amplicons for use in SV40, JC or BK determination consisting of ScSV40 primer, ScJC primer, ScBK primer, Sc SV40 probe, Sc JC probe, ScBK probe, SV40 P1, SV40 P2, SV40 P3, SV40 P4, JC P1, JC P2, JC P3, JC P4, BK P1, BK P2, BK P3, or BK P4, and any mixture thereof.

57. A self-probing amplicon according to claim 1, further including a tail region comprising SEQ ID NO: 10.

58. A self-probing amplicon according to claims 1 or 2, further including a probe component adapted to bind to the GP6+ sequence.

59. A self-probing amplicon comprising a nucleic acid sequence comprising:
(a) a primer component; and
(b) a probe component;
wherein the primer component comprises a sequence selected from the group consisting of SEQ ID NOS: 32 to 40, SEQ ID NOS: 47 to 52, or SEQ ID NO: 61; and
wherein SEQ ID NO: 61 is a primer component of SEQ ID NOS: 19-20.

60. A self-probing amplicon comprising a nucleic acid sequence comprising:
(a) a primer component; and
(b) a probe component;
wherein the probe component comprises a sequence selected from the group consisting of SEQ ID NOS: 21 to 29, SEQ ID NO: 31, SEQ ID NOS: 41 to 46, or SEQ IDS NOS: 59-60; and
wherein SEQ ID NOS:59-60 are the probe component of SEQ ID NOS: 19-20.

61. A diagnostic kit for the detection, typing, or determination of viral cell load or viral integration state comprising:
(a) at least one virus self-probing amplicon, housekeeping self-probing amplicon, or tailed primer; and
(b) instructions for use of the kit.

62. A kit for estimating viral load per cell according to claim 6, comprising at least two virus self-probing amplicons.

63. A kit according to claim 6 or claim 7, comprising at least one housekeeping self-probing amplicon.


SEQUENCE LISTING (61 sequences)

SEQ ID NO: 1 - DNA, 54 bp, Homo sapiens
ccgcggataaagagtacatgcgtccgcgggaaaaataaattgtaaatcatactc

SEQ ID NO: 2 - DNA, 60 bp, Homo sapiens
cccgcggcagattataaggaatacatgcccgcggggaaaaataaactgtaaatcaaactc

SEQ ID NO: 3 - DNA, 54 bp, Homo sapiens
ccgcggagtacctacgacatgggccgcgggaaaaataaactgtaaatcatattc

SEQ ID NO: 4 - DNA, 57 bp, Homo sapiens
ccgcggcagcagtatagcagacatggccgcgggaaaaataaactgcaaatcatattc

SEQ ID NO: 5 - DNA, 58 bp, Homo sapiens
cccgcgggagtatttaagacatggtgccgcggggaaatataaattgtaaatcaaattc

SEQ ID NO: 6 - DNA, 55 bp, Homo sapiens
ccgcggctttatgcacacaagaacccgcgggaaaaacaaactgtagatcatattc

SEQ ID NO: 7 - DNA, 56 bp, Homo sapiens
cccgcggaatataccaggcacgtgccgcggggaaatataaattgtaaatcatactc

SEQ ID NO: 8 - DNA, 57 bp, Homo sapiens
cccgcgggcaatatattaggcatgggccgcgggaaaaataaattgcaattcatactc

SEQ ID NO: 9 - DNA, 56 bp, Homo sapiens
ccgcggtcagtaccttagacatgtgccgcgggaaaaacaaattgtaacccatattc

SEQ ID NO: 10 - DNA, 18 bp, Homo sapiens
atgtggaaacatgcatgg

SEQ ID NO: 11 - DNA, 48 bp, Homo sapiens
ccgcggatggtgtctgtttgagccgcggacacaactgtgttcactagc

SEQ ID NO: 12 - DNA, 54 bp, Homo sapiens
ccgcgggcaaagagtaatcattaccgcggcagaatggatacaaagacaaacagt

SEQ ID NO: 13 - DNA, 63 bp, Homo sapiens
cccgcggttgtcatatagacatatcattttcatccgcgggcaacgtttaaatgtgtgtcagga

SEQ ID NO: 14 - DNA, 60 bp, Homo sapiens
ccgctggcgaatgtctacatatcatggcccagcggaagttaccacagttatgcacagagc

SEQ ID NO: 15 - DNA, 54 bp, Homo sapiens
cccgcggtcggtgtctccatgttgccgcgggagtaatgggagacacacctgagt

SEQ ID NO: 16 - DNA, 53 bp, Homo sapiens
ccgcggtacattgtcatggtctatgatccgcgggcagacaccgaaggaaaccc

SEQ ID NO: 17 - DNA, 55 bp, Homo sapiens
ccgctggctggaatgctatatcatgccagcggacccagaaagttaccacagttat

SEQ ID NO: 18 - DNA, 49 bp, Homo sapiens
atgtggaaacatgcatggcgacatgaaaaataaactgtaaatcatatct

SEQ ID NO: 19 - DNA, 49 bp, Homo sapiens
ccgcgggaagaatatgatttacaccgcgggtggaaacatgcatggcgac

SEQ ID NO: 20 - DNA, 49 bp, Homo sapiens
ccgcgggaggaatatgatttacaccgcgggtggaaacatgcatggcgac

SEQ ID NO: 21 - DNA, 17 bp, Homo sapiens
ataaagagtacatgcgt

SEQ ID NO: 22 - DNA, 21 bp, Homo sapiens
cagattataaggaatacatgc

SEQ ID NO: 23 - DNA, 17 bp, Homo sapiens
agtacctacgacatggg

SEQ ID NO: 24 - DNA, 18 bp, Homo sapiens
agcagtatagcagacatg

SEQ ID NO: 25 - DNA, 19 bp, Homo sapiens
gagtatttaagacatggtg

SEQ ID NO: 26 - DNA, 18 bp, Homo sapiens
ctttatgcacacaagaac

SEQ ID NO: 27 - DNA, 17 bp, Homo sapiens
aatataccaggcacgtg

SEQ ID NO: 28 - DNA, 19 bp, Homo sapiens
gcaatatattaggcatggg

SEQ ID NO: 29 - DNA, 19 bp, Homo sapiens
tcagtaccttagacatgtg

SEQ ID NO: 30 - skipped

SEQ ID NO: 31 - DNA, 16 bp, Homo sapiens
atggtgtctgtttgag

SEQ ID NO: 32 - DNA, 25 bp, Homo sapiens
gaaaaataaattgtaaatcatactc

SEQ ID NO: 33 - DNA, 25 bp, Homo sapiens
gaaaaataaactgtaaatcaaactc

SEQ ID NO: 34 - DNA, 25 bp, Homo sapiens
gaaaaataaactgtaaatcatattc

SEQ ID NO: 35 - DNA, 25 bp, Homo sapiens
gaaaaataaactgcaaatcatattc

SEQ ID NO: 36 - DNA, 25 bp, Homo sapiens
gaaatataaattgtaaatcaaattc

SEQ ID NO: 37 - DNA, 25 bp, Homo sapiens
gaaaaacaaactgtagatcatattc

SEQ ID NO: 38 - DNA, 25 bp, Homo sapiens
gaaatataaattgtaaatcatactc

SEQ ID NO: 39 - DNA, 24 bp, Homo sapiens
aaaaataaattgcaattcatactc

SEQ ID NO: 40 - DNA, 25 bp, Homo sapiens
gaaaaacaaattgtaacccatattc

SEQ ID NO: 41 - DNA, 17 bp, Homo sapiens
gcaaagagtaatcatta

SEQ ID NO: 42 - DNA, 26 bp, Homo sapiens
ttgtcatatagacatatcattttcat

SEQ ID NO: 43 - DNA, 21 bp, Homo sapiens
cgaatgtctacatatcatggc

SEQ ID NO: 44 - DNA, 17 bp, Homo sapiens
tcggtgtctccatgttg

SEQ ID NO: 45 - DNA, 21 bp, Homo sapiens
tacattgtcatggtctatgat

SEQ ID NO: 46 - DNA, 18 bp, Homo sapiens
ctggaatgctatatcatg

SEQ ID NO: 47 - DNA, 25 bp, Homo sapiens
cagaatggatacaaagacaaacagt

SEQ ID NO: 48 - DNA, 23 bp, Homo sapiens
caacgtttaaatgtgtgtcagga

SEQ ID NO: 49 - DNA, 25 bp, Homo sapiens
aagttaccacagttatgcacagagc

SEQ ID NO: 50 - DNA, 23 bp, Homo sapiens
agtaatgggagacacacctgagt

SEQ ID NO: 51 - DNA, 20 bp, Homo sapiens
gcagacaccgaaggaaaccc

SEQ ID NO: 52 - DNA, 23 bp, Homo sapiens
acccagaaagttaccacagttat

SEQ ID NO: 53 - DNA, 17 bp, Homo sapiens
accccaaggactttcct

SEQ ID NO: 54 - DNA, 19 bp, Homo sapiens
cctatggaacagatgaatg

SEQ ID NO: 55 - DNA, 17 bp, Homo sapiens
accctaaagactttccc

SEQ ID NO: 56 - DNA, 28 bp, Homo sapiens
agcatgactcaaaaaacttagcaattct

SEQ ID NO: 57 - DNA, 28 bp, Homo sapiens
ttctcattaaatgtattccaccaggatt

SEQ ID NO: 58 - DNA, 28 bp, Homo sapiens
agcttgactaagaaactggtgtagatca

SEQ ID NO: 59 - DNA, 17 bp, Homo sapiens
gaagaatatgatttaca

SEQ ID NO: 60 - DNA, 17 bp, Homo sapiens
gaggaatatgatttaca

SEQ ID NO: 61 - DNA, 20 bp, Homo sapiens
gtggaaacatgcatggcgac